(19) United States
(12) Patent Application Publication
(10) Pub. No.: US 2018/0237810 A1
(43) Pub. Date: Aug. 23, 2018

(54) RECOMBINANT HOST CELLS FOR THE PRODUCTION OF MALONATE

(71) Applicant: Lygos, Inc., Emeryville, CA (US)

(72) Inventor: Jeffrey A. Dietrich, San Francisco, CA (US)

(21) Appl. No.: 15/321,662

(22) PCT Filed: Jun. 24, 2015
(86) PCT No.: PCT/US2015/037530

§ 371 Date: Dec. 22, 2016

Related U.S. Application Data
(60) Provisional application No. 62/017,723, filed on Jun. 26, 2014.

(51) Int. Cl. C12P 7/46; C07K 14/38; C07K 14/37; C12N 15/81

(52) U.S. Cl. CPC C12P 7/46; C12N 15/81; C07K 14/37; C07K 14/38

(57) ABSTRACT

Systems and methods for the production of malonate in recombinant host cells.

### BACKGROUND OF THE INVENTION

The long-term economic and environmental concerns associated with the petrochemical industry has provided the impetus for increased research, development, and commercialization of processes for conversion of carbon feedstocks into chemicals that can replace those petroleum feedstocks. One approach is the development of biorefining processes to convert renewable feedstocks into products that can replace petroleum-derived chemicals. Two common goals in improving a biorefining process include achieving a lower cost of production and reducing carbon dioxide emissions.

Propanedioic acid (“malonate”, CAS No. 141-82-2) is currently produced from non-renewable, petroleum feedstocks. Mono- or di-esterification of one or both carboxylic acid moieties of malonate with an alcohol (e.g. methanol or ethanol) yields the monoalkyl and dialkyl malonates, respectively. 2,2-dimethyl-1,3-dioxane-4,6-dione (“Meldrum's acid” CAS No. 2033-24-1) is produced from malonate using either acetone in acetic anhydride or isopropenyl acetate in acid.

Chemical synthesis is currently the preferred route for synthesis of malonate and malonate derived compounds. For example, dialkyl malonates are produced through either a hydrogen cyanide or carbon monoxide process. In the hydrogen cyanide process, sodium cyanide is reacted with sodium chloroacetate at elevated temperatures to produce sodium cyanoacetate, which is subsequently reacted with an alcohol/mineral acid mixture to produce the dialkyl malonate. Hildbrand et al. report yields of 75-85% (see “Malonic acid and Derivatives” In: Ullmann's Encyclopedia of Industrial Chemistry, Wiley-VCH, Weinheim, New York (2002)). In the carbon monoxide process, dialkyl malonates (also referred to herein as diester malonates) are produced through cobalt-catalyzed alkoxycarbonylation of chloroacetates with carbon monoxide in the presence of an alcohol at elevated temperatures and pressures.

The existing, petrochemical-based production routes to the malonate and malonate-derived compounds are low yielding, environmentally damaging, dependent upon non-renewable feedstocks, and require expensive treatment of wastewater and exhaust gas. Recently, new methods for producting malonate using biological processes have been described (see PCT Pub. No. WO 13/134424, incorporated herein by reference). There remains a need, however, for improved methods and materials for biocatalytic conversion of renewable feedstocks into malonate, purification of biosynthetic malonate, and subsequent preparation of downstream chemicals and products.

### SUMMARY OF THE INVENTION

The present invention relates to compositions and methods for producing malonate in recombinant host cells. In accordance with the present invention, increased malonate titer, yield, and/or productivity can be achieved by genetic modifications that increase production of malonate by the host cell, and the invention provides recombinant host cells comprising nucleic acids encoding MAE1 transport proteins that increase production of malonate by the host cell and vectors for expressing MAE1 transport proteins that increase production of malonate by the host cell. The invention also provides methods for the use of recombinant host cells comprising MAE1 transport proteins for the production of malonate.

In a first aspect, the invention provides a recombinant host cell capable of producing malonate comprising a heterologous nucleic acid encoding a malic acid transport protein (herein referred to as MAE1 transport protein). In one embodiment, the recombinant host cell has been engineered to produce malonate (e.g., as per methods described in PCT Pub. No. WO 13/134424, supra). In another embodiment, the recombinant host cell natively produces malonate. These recombinant host cells produce more malonate than counterpart cells that do not comprise such a MAE1 transport protein. In various embodiments, the host cells can produce at least 1.5-fold more malonate under appropriate fermentation conditions relative to parental or control cells that do not comprise a heterologous nucleic acid encoding a MAE1 transport protein. In some embodiments, the recombinant host cell is a yeast cell. In one embodiment, the recombinant host cell is a Pichia kudriavzevii cell.

In various embodiments, the heterologous nucleic acid provided by the invention encodes a MAE1 transport protein. Suitable MAE1 transport proteins can be obtained from various eukaryotic organisms. In various embodiments, the MAE1 transport protein is obtained from an Aspergillus species or a Schizosaccharomyces species. Various constructs of the invention utilize the Aspergillus niger A2R8T9 MAE1 transport protein sequence (SEQ ID NO: 1) or variants of it. Thus, in various embodiments, suitable MAE1 transport proteins for use in the methods of the invention have at least 25%, at least 50%, at least 75%, at least 95%, or at least 99% identity to SEQ ID NO: 1. Other MAE1 transport proteins are also suitable for use in accordance with the methods of the invention, and in various embodiments recombinant host cells capable of producing malonate comprise heterologous nucleic acids encoding MAE1 transporters with at least 25%, at least 60%, at least 80%, at least 90%, at least 95%, or more than 95% sequence identity to Aspergillus kawachi G7XR17 (SEQ ID NO: 2) and/or Aspergillus terreus Q0D1U9 (SEQ ID NO: 3) MAE1 transport proteins.

The invention also provides a variety of recombinant host cells comprising heterologous nucleic acids encoding MAE1 transport proteins homologous to MAE1 transport protein consensus sequences contained herein. These consensus consequences are broadly useful for determining if a putative transport protein is an MAE1 transport protein suitable for use in accordance with the methods of the invention. In various embodiments, recombinant host cells capable of producing malonate comprise heterologous nucleic acids encoding MAE1 transporters with at least 45% sequence identity to Aspergillus MAE1 consensus sequence (SEQ ID NO: 7). In some embodiments, recombinant host cells capable of producing malonate comprise heterologous nucleic acids encoding MAE1 transporters with at least 80% sequence identity to Aspergillus MAE1 consensus sequence (SEQ ID NO: 7).

In a second aspect, the invention provides recombinant expression vectors encoding a MAE1 transport protein that increase production of malonate by the host cell. In some embodiments, the expression vector is a yeast expression vector. In various embodiments, the expression vector is a Pichia kudriavzevii expression vector. In other embodiments, the expression vector is a Saccharomyces cerevisiae expression vector.

In a third aspect, the invention provides methods for producing malonate in a recombinant host cell, which methods generally comprise culturing the recombinant host cell capable of producing malonate and comprising a heterologous nucleic acid encoding a MAE1 transport protein under conditions that enable the recombinant host cell to produce malonate.

These and other aspects and embodiments of the invention are described in more detail below.

### DETAILED DESCRIPTION OF THE INVENTION

The present invention relates to compositions and methods for producing malonate in recombinant host cells. In accordance with the present invention, increased malonate titer, yield, and/or productivity can be achieved by genetic modifications that increase production of malonate by the host cell, and the invention provides recombinant host cells comprising nucleic acids encoding MAE1 transport proteins that increase production of malonate by the host cell and vectors for expressing MAE1 transport proteins that increase production of malonate by the host cell. The invention also provides methods for the use of recombinant host cells comprising MAE1 transport proteins for the production of malonate.

While the present invention is described herein with reference to aspects and specific embodiments thereof, those skilled in the art will recognize that various changes may be made and equivalents may be substituted without departing from the invention. The present invention is not limited to particular nucleic acids, expression vectors, enzymes, host microorganisms, or processes, as such may vary. The terminology used herein is for purposes of describing particular aspects and embodiments only, and is not to be construed as limiting. In addition, many modifications may be made to adapt a particular situation, material, composition of matter, process, process step or steps, in accordance with the invention. All such modifications are within the scope of the claims appended hereto.

All patents, patent applications, and publications cited herein are incorporated herein by reference in their entireties.

### Definitions

In this specification and in the claims that follow, reference will be made to a number of terms that shall be defined to have the following meanings.

As used in the specification and the appended claims, the singular forms “a,” “an,” and “the” include plural referents unless the context clearly dictates otherwise. Thus, for example, reference to an “expression vector” includes a single expression vector as well as a plurality of expression vectors, either the same (e.g., the same operon) or different; reference to “cell” includes a single cell as well as a plurality of cells; and the like.

Amino acids in the sequence listing are identified by a three-letter abbreviation, as follows: Ala is alanine, Arg is arginine, Asn is asparagine, Asp is aspartic acid, Cys is cysteine, Gln is glutamine, Glu is glutamic acid, Gly is glycine, His is histidine, Leu is leucine, Ile is isoleucine, Lys is lysine, Met is methionine, Phe is phenylalanine, Pro is proline, Ser is serine, Thr is threonine, Trp is tryptophan, Tyr is tyrosine, and Val is valine. At some positions, Xaa indicates that any amino acid may be present at the specified position. At other positions, Xaa indicates that one of a subset of amino acids can be present, namely Xaa may represent Arg, Lys, His, Asp, Glu, Ile, Lys, Val, Ser, or Thr at the indicated position.

Specific amino acid in protein coding sequences discussed herein are identified by their respective single-letter abbreviation, as follows: A is alanine, R is arginine, N is asparagine, D is aspartic acid, C is cysteine, Q is glutamine, E is glutamic acid, G is glycine, H is histidine, L is leucine, I is isoleucine, L is lysine, M is methionine, F is phenylalanine, P is proline, S is serine, T is threonine, W is tryptophan, Y is tyrosine, and V is valine. In some instances, these single-letter abbreviations are followed by the amino acid position in the protein coding sequence where 1 corresponds to the amino acid (typically methionine) at the N-terminus of the protein. For example, E124 in S. cerevisiae wild type EHD3 refers to the glutamic acid at position 124 from the EHD3 N-terminal methionine (i.e., M1). Amino acid substitutions (i.e., point mutations) are indicated by identifying the mutated (i.e., progeny) amino acid after the single-letter code and number in the parental protein coding sequence; for example, E124A in S. cerevisiae EHD3 refers to substitution of alanine for glutamic acid at position 124 in the EHD3 protein coding sequence. The mutation may also be identified in parentheticals, for example EHD3 (E124A). Multiple point mutations in the protein coding sequence are separated by a backslash (/); for example, EHD3 E124A/Y125A indicates that mutations E124A and Y125A are both present in the EHD3 protein coding sequence. The number of mutations introduced into some examples has been annotated by a dash followed by the number of mutations, preceding the parenthetical identification of the mutation (e.g. A5W8H3-1 (E95Q)). The UniProt IDs with and without the dash and number are used interchangeably herein (i.e. A5W8H3-1 (E95Q)=A5W8H3 (E95Q)).

As used herein, the term “express”, when used in connection with a nucleic acid a protein or a protein itself in a cell, means that the protein, which may be an endogenous or exogenous (heterologous) protein, is produced in the cell. The term “overexpress”, in these contexts, means that the protein is produced at a higher level, i.e., protein levels are increased, as compared to the wild type, in the case of an endogenous protein. Those skilled in the art appreciate that overexpression of a protein can be achieved by increasing the strength or changing the type of the promoter used to drive expression of a coding sequence, increasing the strength of the ribosome binding site or Kozak sequence, increasing the stability of the mRNA transcript, altering the codon usage, increasing the stability of the protein, and the like.

The terms “expression vector” or “vector” refer to a nucleic acid and/or a composition comprising a nucleic acid that can be introduced into a host cell, e.g., by transduction, transformation, or infection, such that the cell then produces (“expresses”) nucleic acids and/or proteins other than those native to the cell, or in a manner not native to the cell, that are contained in or encoded by the nucleic acid so introduced. Thus, an “expression vector” contains nucleic acids (ordinarily DNA) to be expressed by the host cell. Optionally, the expression vector can be contained in materials to aid in achieving entry of the nucleic acid into the host cell, such as the materials associated with a virus, liposome, protein coating, or the like. Expression vectors suitable for use in various aspects and embodiments of the present invention include those into which a nucleic acid sequence can be, or has been, inserted, along with any preferred or required operational elements. Thus, an expression vector can be transferred into a host cell and, typically, replicated therein (although, one can also employ, in some embodiments, non-replicable vectors that provide for “transient” expression). In some embodiments, an expression vector that integrates into chromosomal, mitochondrial, or plastid DNA is employed. In other embodiments, an expression vector that replicates extrachromasomally is employed. Typical expression vectors include plasmids, and expression vectors typically contain the operational elements required for transcription of a nucleic acid in the vector. Such plasmids, as well as other expression vectors, are described herein or are well known to those of ordinary skill in the art.

The terms “ferment”, “fermentative”, and “fermentation” are used herein to describe culturing microbes under conditions to produce useful chemicals, including but not limited to conditions under which microbial growth, be it aerobic or anaerobic, occurs.

The term “heterologous” as used herein refers to a material that is non-native to a cell. For example, a nucleic acid is heterologous to a cell, and so is a “heterologous nucleic acid” with respect to that cell, if at least one of the following is true: (a) the nucleic acid is not naturally found in that cell (that is, it is an “exogenous” nucleic acid); (b) the nucleic acid is naturally found in a given host cell (that is, “endogenous to”), but the nucleic acid or the RNA or protein resulting from transcription and translation of this nucleic acid is produced or present in the host cell in an unnatural (e.g., greater or lesser than naturally present) amount; (c) the nucleic acid comprises a nucleotide sequence that encodes a protein endogenous to a host cell but differs in sequence from the endogenous nucleotide sequence that encodes that same protein (having the same or substantially the same amino acid sequence), typically resulting in the protein being produced in a greater amount in the cell, or in the case of an enzyme, producing a mutant version possessing altered (e.g. higher or lower or different) activity; and/or (d) the nucleic acid comprises two or more nucleotide sequences that are not found in the same relationship to each other in the cell. As another example, a protein is heterologous to a host cell if it is produced by translation of RNA or the corresponding RNA is produced by transcription of a heterologous nucleic acid; a protein is also heterologous to a host cell if it is a mutated version of an endogenous protein, and the mutation was introduced by genetic engineering.

The terms “host cell” and “host microorganism” are used interchangeably herein to refer to a living cell that can be (or has been) transformed via insertion of an expression vector. A host microorganism or cell as described herein may be a prokaryotic cell (e.g., a microorganism of the kingdom Eubacteria) or a eukaryotic cell. As will be appreciated by one of skill in the art, a prokaryotic cell lacks a membrane-bound nucleus, while a eukaryotic cell has a membrane-bound nucleus.

The terms “isolated” or “pure” refer to material that is substantially, e.g. greater than 50% or greater than 75%, or essentially, e.g. greater than 90%, 95%, 98% or 99%, free of components that normally accompany it in its native state, e.g. the state in which it is naturally found or the state in which it exists when it is first produced.

A carboxylic acid as described herein can be a salt, acid, base, or derivative depending on the structure, pH, and ions present. The terms “malonate” and “malonic acid” are used interchangeably herein. Malonic acid is also called propanedioic acid (C₃H₄O₄; CAS# 141-82-2).

The term “malonate-derived compounds” as used herein refers to mono-alkyl malonate esters, including, for example and without limitation, mono-methyl malonate (also referred to as monomethyl malonate, CAS# 16695-14-0), mono-ethyl malonate (also referred to as monoethyl malonate, CAS# 1071-46-1), mono-propyl malonate, mono-butyl malonate, mono-tert-butyl malonate (CAS# 40052-13-9), and the like; di-alkyl malonate esters, for example and without limitation, dimethyl malonate (CAS# 108-59-8), diethyl malonate (CAS# 105-53-3), dipropyl malonate (CAS# 1117-19-7), dibutyl malonate (CAS# 1190-39-2), and the like, and Meldrum' s acid (CAS# 2033-24-1). The malonate-derived compounds can be produced synthetically from malonate and are themselves valuable compounds but are also useful substrates in the chemical synthesis of a number of other valuable compounds.

As used herein, the term “nucleic acid” and variations thereof shall be generic to polydeoxyribonucleotides (containing 2-deoxy-D-ribose) and to polyribonucleotides (containing D-ribose). “Nucleic acid” can also refer to any other type of polynucleotide that is an N-glycoside of a purine or pyrimidine base, and to other polymers containing nonnucleotidic backbones, provided that the polymers contain nucleobases in a configuration that allows for base pairing and base stacking, as found in DNA and RNA. As used herein, the symbols for nucleotides and polynucleotides are those recommended by the IUPAC-IUB Commission of Biochemical Nomenclature (Biochem. 9:4022, 1970). A “nucleic acid” may also be referred to herein with respect to its sequence, the order in which different nucleotides occur in the nucleic acid, as the sequence of nucleotides in a nucleic acid typically defines its biological activity, e.g., as in the sequence of a coding region, the nucleic acid in a gene composed of a promoter and coding region, which encodes the product of a gene, which may be an RNA, e.g. a rRNA, tRNA, or mRNA, or a protein (where a gene encodes a protein, both the mRNA and the protein are “gene products” of that gene).

The term “operably linked” refers to a functional linkage between a nucleic acid expression control sequence (such as a promoter, ribosome-binding site, and transcription terminator) and a second nucleic acid sequence, the coding sequence or coding region, wherein the expression control sequence directs or otherwise regulates transcription and/or translation of the coding sequence.

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

As used herein, “recombinant” refers to the alteration of genetic material by human intervention. Typically, recombinant refers to the manipulation of DNA or RNA in a cell or virus or expression vector by molecular biology (recombinant DNA technology) methods, including cloning and recombination. Recombinant can also refer to manipulation of DNA or RNA in a cell or virus by random or directed mutagenesis. A “recombinant” cell or nucleic acid can typically be described with reference to how it differs from a naturally occurring counterpart (the “wild-type”). In addition, any reference to a cell or nucleic acid that has been “engineered” or “modified” and variations of those terms, is intended to refer to a recombinant cell or nucleic acid.

The terms “transduce”, “transform”, “transfect”, and variations thereof as used herein refers to the introduction of one or more nucleic acids into a cell. For practical purposes, the nucleic acid must be stably maintained or replicated by the cell for a sufficient period of time to enable the function(s) or product(s) it encodes to be expressed for the cell to be referred to as “transduced”, “transformed”, or “transfected”. As will be appreciated by those of skill in the art, stable maintenance or replication of a nucleic acid may take place either by incorporation of the sequence of nucleic acids into the cellular chromosomal DNA, e.g., the genome, as occurs by chromosomal integration, or by replication extrachromosomally, as occurs with a freely-replicating plasmid. A virus can be stably maintained or replicated when it is “infective”: when it transduces a host microorganism, replicates, and (without the benefit of any complementary virus or vector) spreads progeny expression vectors, e.g., viruses, of the same type as the original transducing expression vector to other microorganisms, wherein the progeny expression vectors possess the same ability to reproduce.

### Recombinant Host Cells

In one aspect, the invention provides a recombinant host cell capable of producing malonate, the host cell comprising a heterologous nucleic acid encoding a malic acid transport protein (herein referred to as a MAE1 transport protein or a MAE1 transporter). In one embodiment, the recombinant host cell has been engineered to produce malonate. In another embodiment, the recombinant host cell natively produces malonate.

The present invention results in part from the discovery that a host cell expressing a MAE1 transport protein results in increased production of malonate relative to a parental host cell that does not express the MAE1 transport protein. Any suitable host cell may be used in practice of the methods of the present invention. In some embodiments, the host cell is a recombinant host microorganism capable of producing malonate that comprises a nucleic acid encoding a MAE1 transport protein that results in expression of the transport protein and provides an increase in the yield, titer, and/or productivity of malonate relative to a “control cell” or “reference cell” that does not express the transport protein, or produces less of it. A “control cell” is thus used for comparative purposes, and can be a recombinant parental cell that does not contain one or more of the modification(s) that result in MAE1 transport protein expression (or increased expression) in the host cell of the invention. Malonate is not naturally produced at high concentrations in naturally occurring microbes (i.e. non-recombinant microbes).

A variety of recombinant host cells are useful in accordance with the methods of the invention. In an important embodiment, the recombinant host cell is a yeast cell. Yeast cells are excellent host cells for construction of recombinant metabolic pathways comprising heterologous enzymes catalyzing production of small molecule products. There are established molecular biology techniques and nucleic acids encoding genetic elements necessary for construction of yeast expression vectors, including, but not limited to, promoters, origins of replication, antibiotic resistance markers, auxotrophic markers, terminators, and the like. Second, techniques for integration of nucleic acids into the yeast chromosome are well established. Yeast also offers a number of advantages as an industrial fermentation host. Yeast cells can tolerate high concentrations of organic acids and maintain cell viability at low pH and can grow under both aerobic and anaerobic culture conditions, and there are established fermentation broths and fermentation protocols. The ability of a strain to propagate and/or produce desired product under low pH provides a number of advantages with regard to the present invention. First, this characteristic provides tolerance to the environment created by the production of malonate. Second, from a process standpoint, the ability to maintain a low pH environment limits the number of organisms that are able to contaminate and spoil a batch.

In various embodiments, yeast cells useful in the method of the invention include yeasts of a genera selected from the non-limiting group consisting of Aciculoconidium, Ambrosiozyma, Arthroascus, Arxiozyma, Ashbya, Babjevia, Bensingtonia, Botryoascus, Botryozyma, Brettanomyces, Bullera, Bulleromyces, Candida, Citeromyces, Clavispora, Cryptococcus, Cystofilobasidium, Debaryomyces, Dekkara, Dipodascopsis, Dipodascus, Eeniella, Endomycopsella, Eremascus, Eremothecium, Erythrobasidium, Fellomyces, Filobasidium, Galactomyces, Geotrichum, Guilliermondella, Hanseniaspora, Hansenula, Hasegawaea, Holtermannia, Hormoascus, Hyphopichia, Issatchenkia, Kloeckera, Kloeckeraspora, Kluyveromyces, Kondoa, Kuraishia, Kurtzmanomyces, Leucosporidium, Lipomyces, Lodderomyces, Malassezia, Metschnikowia, Mrakia, Myxozyma, Nadsonia, Nakazawaea, Nematospora, Ogataea, Oosporidium, Pachysolen, Phachytichospora, Phaffia, Pichia, Rhodosporidium, Rhodotorula, Saccharomyces, Saccharomycodes, Saccharomycopsis, Saitoella, Sakaguchia, Saturnospora, Schizoblastosporion, Schizosaccharomyces, Schwanniomyces, Sporidiobolus, Sporobolomyces, Sporopachydermia, Stephanoascus, Sterigmatomyces, Sterigmatosporidium, Symbiotaphrina, Sympodiomyces, Sympodiomycopsis, Torulaspora, Trichosporiella, Trichosporon, Trigonopsis, Tsuchiyaea, Udeniomyces, Waltomyces, Wickerhamia, Wickerhamiella, Williopsis, Yamadazyma, Yarrowia, Zygoascus, Zygosaccharomyces, Zygowilliopsis, and Zygozyma, among others.

In various embodiments, the yeast cell is of a species selected from the non-limiting group consisting of Candida albicans, Candida ethanolica, Candida guilliermondii, Candida krusei, Candida lipolytica, Candida methanosorbosa. Candida sonorensis, Candida tropicalis, Candida utilis, Cryptococcus curvatus, Hansenula polymorpha, Issatchenkia orientalis, Kluyveromyces lactis, Kluyveronmyces marxianus, Kluyveromyces thermotolerans, Komagataella pastoris, Lipomyces starkeyi, Pichia angusta, Pichia deserticola, Pichia galeiformis, Pichia kodarnae, Pichia kudriavzevii, Pichia membranaefaciens, Pichia methanolica, Pichia pastoris, Pichia salictaria, Pichia stipitis, Pichia thermotolerans, Pichia trehalophfla, Rhodosporidium toruloides, Rhodotorula glutinis, Rhodotorula graminis, Saccharomyces bayanus, Saccharomyces boulardi, Saccharomyces cerevisiae, Saccharomyces kluyveri, Schizosaccharomyces pombe and Yarrowia lipolytica. One skilled in the art will recognize that this list encompasses yeast in the broadest sense, including both oleaginous and non-oleaginous strains.

In certain embodiments, the recombinant yeast cells provided herein are engineered by the introduction of one or more genetic modifications (including, for example, introduction of heterologous nucleic acids encoding MAE1 transport proteins) into a Crabtree-negative yeast cell. As used herein, “a Crabtree-negative yeast cell” refers to a yeast cell that does not undergo immediate aerobic alcohol fermentation in response to addition of excess sugar following growth under sugar-limited conditions. In certain of these embodiments, the host cell belongs to the Pichia/Issatchenkia/Saturnispora/Dekkera Glade. In certain of these embodiments, the host cell belongs to the genus selected from the group consisting of Pichia, Issatchenkia, or Candida. In certain embodiments, the host cell belongs to the genus Pichia. In one embodiment, the recombinant host cell is a Pichia kudriavzevii host cell. Examples 1 and 2, below, illustrate the use of Pichia kudriavzevii in accordance with the invention.

In certain embodiments, the recombinant host cells provided herein are engineered by introduction of one or more genetic modifications into a Crabtree-positive yeast cell. As used herein, “a Crabtree-positive yeast cell” refers to a yeast cell that undergoes immediate aerobic alcohol fermentation in response to addition of excess sugar following growth under sugar-limited conditions. In certain of these embodiments, the host cell belongs to the Saccharomyces Glade. In certain of these embodiments, the host cell belongs to a genus selected from the group consisting of Saccharomyces, Hanseniaspora, and Kluyveromyces. In certain embodiments, the host cell belongs to the genus Saccharomyces. In one embodiment, the host cell is Sachcharomyces kluyveri. In another embodiment, the recombinant host cell is a Saccharomyces cerevisiae host cell.

Members of the Pichia/Issatchenkia/Saturnispora/Dekkera or the Saccharomyces Glade are identified by analysis of their 26S ribosomal DNA using the methods described by Kurtzman C.P., and Robnett C.J., (“Identification and Phylogeny of Ascomycetous Yeasts from Analysis of Nuclear Large Subunit (26S) Ribosomal DNA Partial Sequences”, Atonie van Leeuwenhoek 73(4):331-371; 1998). Kurtzman and Robnett report analysis of approximately 500 ascomycetous yeasts were analyzed for the extent of divergence in the variable D1/D2 domain of the large subunit (26S) ribosomal DNA. Host cells encompassed by a Glade exhibit greater sequence identity in the D 1/D2 domain of the 26S ribosomal subunit DNA to other host cells within the Glade as compared to host cells outside the Glade. Therefore, host cells that are members of a Glade (e.g., the Pichia/Issatchenkia/Saturnispora/Dekkera or Saccharomyces clades) can be identified using the methods of Kurtzman and Robnett.

Recombinant host cells other than yeast cells are also suitable for use in accordance with the methods of the invention. Illustrative examples include various eukaryotic, prokaryotic, and archaeal host cells. Illustrative examples of eukaryotic host cells provided by the invention include, but are not limited to cells belonging to the genera Aspergillus, Crypthecodinium, Cunninghamella, Entomophthora, Mortierella, Mucor, Neurospora, Pythium, Schizochytrium, Thraustochytrium Trichoderma, Xanthophyllomyces. Examples of eukaryotic strains include, but are not limited to: Aspergillus niger, Aspergillus oryzae, Crypthecodiniurri cohnii, Cunninghamella japonica, Entomophthora coronata, Mortierella alpina, Mucor circinelloides, Neurospora crassa, Pythium ultimum, Schizochytrium limacinum, Thraustochytrium aureurri, Trichoderma reesei, and Xanthophyllomyces dendrorhous.

Illustrative examples of recombinant archaea host cells provided by the invention include, but are not limited to, cells belonging to the genera: Aeropyrum, Archaeglobus, Halobacterium, Methanococcus, Methanobacterium, Pyrococcus, Sulfolobus, and Thermoplasma. Examples of archae strains include, but are not limited to Archaeoglobus fulgidus, Halobacterium sp., Methanococcus jannaschii, Methanobacterium thermoautotrophicum, Thermoplasma acidophilum, Thermoplasma volcanium, Pyrococcus horikoshii, Pyrococcus abyssi, and Aeropyrum pernix.

Illustrative examples of recombinant prokaryotic host cells provided by the invention include, but are not limited to, cells belonging to the genera Agrobacterium, Alicyclobacillus, Alnabaena, Anacystis, Arthrobacter, Azobacter, Bacillcus, Brevibacterium, Chromatium, Clostridium, Corynebacterium, Enterobacter, Erwinia, Escherichia, Lactobacillus, Lactococcus, Mesorhizobium, Methylobacterium, Microbacterium, Phomndium, Pseudomonas, Rhodobacter, Rhodopseudomonas, Rhodospirillum, Rhodococcus, Salmonella, Scenedesmun, Serratia, Shigella, Staphiococcus, Strepromyces, Synnecoccus, and Zymomonas. Examples of prokaryotic strains include, but are not limited to Bacillus subtilis, Brevibacterium ammoniagenes, Bacillus arnyloliquefacines, Brevibacterium ammoniagenes, Brevibacterium immariophilum, Clostridium beigerinckii, Enterobacter sakazakii, Escherichia coli, Lactobacillus acidophilus, Lactococcus lactis, Mesorhizobium loci, Pseudomonas aeruginosa, Pseudomonas mevalonii, Pseudomonas pudita, Rhodobacter capsulatus, Rhodobacter sphaeroides, Rhodospirillum mbrum, Salmonella enterica, Salmonella typhi, Salmonella typhimurium, Shigella dysenteriae, Shigella flexneri, Shigella sonnei, and Staphylococcus aureus.

Escherichia coli is a particularly good prokaryotic host cell for use in accordance with the methods of the invention. E. coli is well utilized in industrial fermentation of small-molecule products and can be readily engineered. Unlike most wild type yeast strains, wild type E. coli can catabolize both pentose and hexose sugars as carbon sources. The present invention provides a wide variety of recombinant E. coli host cells suitable for use in the methods of the invention. In one embodiment, the recombinant host cell is an Escherichia coli host cell.

Certain of these host cells, including Saccharomyces cerevisiae, Bacillus subtilis, Lactobacillus acidophilus, have been designated by the Food and Drug Administration as Generally Regarded As Safe (or GRAS) and so are employed in various embodiments of the methods of the invention. While desirable from public safety and regulatory standpoints, GRAS status does not impact the ability of a host strain to be used in the practice of this invention; hence, non-GRAS and even pathogenic organisms are included in the list of illustrative host strains suitable for use in the practice of this invention.

### MAE1 Transport Proteins

In accordance with the present invention, certain MAE1 transport proteins have the capacity to transport malonate and increase malonate production in host cells, including naturally occurring host cells but especially recombinant host cells engineered to produce malonate (as per PCT Pub. No. WO 13/134424, supra). An MAE1 transport protein may secrete malonate in an ionic form and in a protonated form. An MAE1 transport protein may transport other species, for example a hydrogen ion, together with malonate. Malonate transport as used herein may be export of malonate from the interior of a cell to the exterior, and/or import of malonate from the exterior to the interior.

As described below, a variety of methods and assays may be used by those skilled in the art to determine if a putative transport protein is a MAE1 transport protein capable of increasing malonate production by a recombinant host cell. For example, the percent sequence identity of a putative MAE1 transport protein relative to a reference MAE1 transport protein sequence is used to determine if a putative transport protein is an MAE1 transport protein. Percent sequence identity is determined by aligning the protein sequence against a reference sequence. The reference sequence can be a consensus sequence or a specific protein sequence. Those skilled in the art will recognize that various sequence alignment algorithms are suitable for aligning a protein with a reference sequence. See, for example, Needleman, S B, et al “A general method applicable to the search for similarities in the amino acid sequence of two proteins.” Journal of Molecular Biology 48 (3): 443-53 (1970). Following alignment of the protein sequence relative to the reference sequence, the percentage of positions where the protein possesses an amino acid (or a dash where no amino acid is present) described by the same position in the reference sequence determines the percent sequence identity. When a degenerate amino acid (represented by Xaa or X) is present in a reference sequence, any of the amino acids described by the degenerate amino acid may be present in the protein at the aligned position for the protein to be identical to the reference sequence at the aligned position.

The Aspergillus niger A2R8T9 MAE1 reference sequence (SEQ ID NO: 1) is useful for determining the percentage sequence identity between a putative MAE1 transport protein and a MAE1 transport protein useful in accordance with the present invention. Suitable MAE1 transport proteins will have at least 25% amino acid sequence identity to SEQ ID NO: 1, and may, for example and without limitation, have at least 50%, 75%, 95%, or greater identity to SEQ ID NO: 1. Thus, proteins G7XR17 (SEQ ID NO: 2, 96% identity), Q0D1U9 (SEQ ID NO: 3, 61% sequence identity), P50537 (SEQ ID NO: 4, 30% sequence identity), and O59815 (SEQ ID NO: 5, 25% sequence identity) have the requisite identity to SEQ ID NO: 1. In contrast, Saccharomyces cerevisiae proteins PDRS (UniProt ID: P33302, 4% identity), PDR10 (UniProt ID: P51533, 4% identity), PDR11 (UniProt ID: P40550, 4% identity), PDR12 (UniProt ID: Q02785, 5% identity), PDR15 (UniProt ID: Q04182, 4% identity), and PDR18 (UniProt ID: P53756, 5% identity) all have less than 25% amino acid identity to SEQ ID NO: 1, and are not MAE1 transport proteins. Example 1 further provides methods for determining if a putative transporter is an MAE1 transport protein and increases malonate production in a recombinant microbe.

Generally, homologous proteins share substantial sequence identity. Any protein substantially homologous to a protein specifically described herein can be used in a host cell of the invention. One protein is homologous to another (the “reference protein”) when it exhibits the same activity of interest and can be used for substantially similar purposes. If a protein shares substantial homology to a reference sequence herein but has suboptimal, including no, MAE1 transport protein activity, then, in accordance with the invention, it can be mutated to conform to a reference sequence provided herein to provide a MAE1 transport protein of the invention.

### Source of MAE1 Transport Proteins

The heterologous nucleic acids encoding a MAE1 transporter may be obtained from microorganisms of any genus. For purposes of the present invention, the term “obtained from” as used herein in connection with a given source shall mean that the MAE1 transporter encoded by a nucleic acid is produced by the source, or by a cell in which the nucleic acid from the source has been inserted. It will be understood that for the organisms indicated below, the invention encompasses taxonomic equivalents (e.g., anamorphs and teleomorphs) regardless of the species name by which they are known. Those skilled in the art will recognize the identity of appropriate equivalents.

In one embodiment, the recombinant host cell capable of producing malonate comprises a nucleic acid encoding a eukaryotic MAE1 transport protein that results in expression of the transport protein and provides an increase in the yield, titer, and/or productivity of malonate relative to a control cell that does not express the transport protein, or produces less of it.

### MAE1 Transport Proteins Obtained from Aspergillus Species or Homologous Thereto

In some embodiments, the recombinant host cell capable of producing malonate comprises a nucleic acid encoding a MAE1 transport protein obtained from an Aspergillus species (or significantly homologous thereto) that results in expression of the transport protein and provides an increase in the yield, titer, and/or productivity of malonate relative to a control cell that does not express the transport protein, or produces less of it. In various embodiments, the nucleic acid encoding a MAE1 transport protein is obtained from an organism selected from the group consisting of, but not limited to, Aspergillus clavatus, Aspergillus flavus, Aspergillus fumigatus, Aspergillus kawachii, Aspergillus nidulans, Aspergillus niger, Aspergillus oryzae, and Aspergillus terreus (or is homologous to such a nucleic acid). In various embodiments, the nucleic acid encodes a MAE1 transport protein selected from the group consisting of Aspergillus niger A2R8T9 (SEQ ID NO: 1), Aspergillus kawachi G7XR17 (SEQ ID NO: 2), and Aspergillus terreus Q0D1U9 (SEQ ID NO: 3). Example 1 demonstrates, in accordance with the methods of the invention, how expression of various MAE1 transport proteins obtained from Aspergillus species (SEQ ID NOs: 1, 2, and 3) by recombinant host cells increases malonate yields and titers relative to parental, control cells that do not express said MAE1 transport proteins. These recombinant host cells have been engineered to express said MAE1 transport proteins through transformation with heterologous nucleic acids encoding the MAE1 transporters. Likewise, Example 3 demonstrates, in accordance with the methods of the invention, how malonate productivity is increased through fermentation of recombinant host cells expressing MAE1 transport proteins obtained from Aspergillus species and that are capable of producing malonate.

In one embodiment, the recombinant host cell capable of producing malonate comprises a nucleic acid encoding an Aspergillus niger A2R8T9 MAE1 transport protein (SEQ ID NO: 1) and provides an increase in the yield, titer, and/or productivity of malonate relative to a control cell that does not express the Aspergillus niger A2R8T9 MAE1 transport protein, or produces less of it. In some embodiments of the invention, the recombinant host cell capable of producing malonate and comprising a nucleic acid encoding an Aspergillus niger A2R8T9 MAE1 transport protein (SEQ ID NO: 1) is a Pichia kudriavzevii host cell. In other embodiments of the invention, the recombinant host cell capable of producing malonate and comprising a nucleic acid encoding an Aspergillus niger A2R8T9 MAE1 transport protein (SEQ ID NO: 1) is a Sacharromyces cerevisiae host cell.

MAE1 transport proteins useful in the compositions and methods provided herein include proteins that are “homologous” to the MAE1 transport proteins obtained from Aspergillus species and described herein. Such homologs have the following characteristics: (1) capable of transporter activity that is identical, or essentially identical, or at least substantially similar with respect to ability to transport malonate across the cell membrane to that of one of the MAE1 transport proteins exemplified herein; (2) shares substantial sequence identity with an MAE1 transport protein described herein; and/or (3) comprises a substantial number of amino acids corresponding to highly conserved amino acids in a MAE1 transport protein described herein.

A “homolog” as used herein refers to a protein that shares substantial sequence identity to a reference protein, such as an MAE1 transport protein, if the amino acid sequence of the homolog is at least 25%, at least 50%, at least 60%, at least 70%, at least 80%, at least 90%, at least 95%, or at least 97% the same as that of an MAE1 transport protein set forth herein.

A number of amino acids in the MAE1 transport proteins provided by the invention are highly conserved across MAE1 transport proteins generally, and proteins homologous to a MAE1 transport protein of the invention will generally possess a substantial number of these highly conserved amino acids. The presence of a highly conserved amino acid in the query protein is determined by first aligning the query protein against the reference sequence; once aligned, the amino acid residue at the highly conserved position in the reference protein is compared to the amino acid residue in the corresponding location in the query protein. If the amino acid residues are the same, then the query protein is said to possess this conserved amino acid. A homolog is said to comprise a substantial number of highly conserved amino acids if at least a majority, often more than 90%, and sometimes all of the highly conserved amino acids are found in the homologous protein.

MAE1 transport proteins suitable for use in accordance with the methods of the invention include those that are homologous to the Aspergillus niger A2R8T9 MAE1 transport protein sequence (SEQ ID NO: 1). In one embodiment, suitable MAE1 transport proteins for use in accordance with the methods of the invention have at least 25% identity to this MAE1 transport protein reference sequence. In other embodiments, suitable MAE1 transport proteins have at least 60% identity to SEQ ID NO: 1. In various embodiments, the MAE1 transport protein has malonate transporter activity and comprises an amino acid sequence having at a percentage sequence identity to SEQ ID NO: 1 of at least 50%, for example, at least 55%, at least 60%, at least 65%, at least 70%, at least 75%, at least 80%, at least 85%, at least 90%, at least 92%, at least 94%, at least 95%, at least 96%, at least 97%, at least 98%, at least 99%, or 100% amino acid sequence identity to SEQ ID NO: 1.

In some embodiments, a MAE1 transport protein with equal to or greater than 25% identity to the reference sequence SEQ ID NO: 1 is expressed in a recombinant host cell capable of producing malonate and is used to increase the production of malonate in said host cell relative the parental host cell. In other embodiments, a MAE1 transport protein with equal to or greater than 60% identity to the reference sequence SEQ ID NO: 1 is expressed in a recombinant host cell capable of producing malonate and is used to increase the production of malonate in said host cell relative the parental host cell. MAE1 proteins possessing substantial sequence homology to SEQ ID NO: 1 and, when expressed in a host cell capable of producing malonate, increase production of malonate include, but are not limited to, G7XR17 (SEQ ID NO: 2; 96% identity), Q0D1U9 (SEQ ID NO: 3; 61% identity), P50537 (SEQ ID NO: 4; 30% identity), and O59815 (SEQ ID NO: 5; 25% identity). As illustrated in Example 1, nucleic acids encoding A2R8T9, G7XR17, Q0D1U9, P50537, and O59815 MAE1 transport proteins were heterologously expressed in a recombinant Pichia kudriavzevii host cell comprising a malonyl-CoA hydrolase and increased malonate titers in the fermentation broth.

As illustrated in Example 2, recombinant expression vectors of the invention comprising nucleic acids encoding the A2R8T9 MAE1 transport protein are heterologously expressed in a genetically modified Pichia kudriavzevii host cell comprising a malonyl-CoA hydrolase and increase malonate productivity. As illustrated in Example 3, fermentation of recombinant host cells capable of producing malonate and expressing MAE1 transport proteins in accordance with the methods of the invention increased malonate productivity.

There are 80 highly conserved amino acids in Aspergillus niger A2R8T9 MAE1 transport protein (SEQ ID NO: 1): R57, H60, F61, T62, W63, W65, M70, G73, G74, F86, G88, L89, R114, F115, I116, E130, F133, T136, L139, I141, T143, I145, L148, L167, I170, F187, T196, P199, L203, P204, F206, P207, M209, G212, I214, A215, Q222, P223, A224, G234, F237, Q238, G239, L240, G241, F242, A250, R255, G260, L261, R267, P268, G269, M270, F271, V274, P276, P277, F279, L282, L284, G299, F320, L324, C330, A332, F344, W348, A350, F353, N355, G357, S371, R398, A399, P408, G409, D411, E412, D413. MAE1 transport proteins homologous to SEQ ID NO: 1 generally possess a majority, often more than 90%, and sometimes all of these highly conserved amino acids. In various embodiments, host cells of invention express a MAE1 transport protein that has at least 95% of these highly conserved amino acids. For example, Q0D1U9 (SEQ ID NO: 3) possess all 80 (i.e., 100%) of the highly conserved amino acids in SEQ ID NO: 1. The location of these amino acids in SEQ ID NO: 3 are as follows (the corresponding location in SEQ ID NO: 1 is provided in parentheses): R34 (R57), H37 (H60), F38 (F61), T39 (T62), W40 (W63), W42 (W65), M47 (M70), G50 (G73), G51 (G74), F63 (F86), G65 (G88), L66 (L89), R91 (R114), F92 (F115), I93 (I116), E107 (E130), F110 (F133), T113 (T136), L116 (L139), I118 (I141), T120 (T143), I122 (I145), L125 (L148), L144 (L167), I147 (I170), F164 (F187), T173 (T196), P176 (P199), L180 (L203), P181 (P204), F183 (F206), P184 (P207), M186 (M209), G189 (G212), I191 (I214), A192 (A215), Q199 (Q222), P200 (P223), A201 (A224), G211 (G234), F214 (F237), Q215 (Q238), G216 (G239), L217 (L240), G218 (G241), F219 (F242), A227 (A250), R232 (R255), G237 (G260), L238 (L261), R244 (R267), P245 (P268), G246 (G269), M247 (M270), F248 (F271), V251 (V274), P253 (P276), P254 (P277), F256 (F279), L259 (L282), L261 (L284), G276 (G299), F297 (F320), L301 (L324), C307 (C330), A309 (A332), F321 (F344), W325 (W348), A327 (A350), F330 (F353), N332 (N355), G334 (G357), 5348 (S371), R375 (R398), A376 (A399), P385 (P408), G386 (G409), D388 (D411), E389 (E412), and D390 (D413). Thus, MAE1 transport protein QOD1U9 has over 95% of the highly conserved amino acids found in SEQ ID NO: 1 and is thus homologous to SEQ ID NO: 1. Other MAE1 transport proteins homologous to SEQ ID NO: 1 include those encoded by the protein sequences set forth in SEQ ID NOs: 1, 2, 4, and 5.

Other MAE1 transport protein sequences in addition to the Aspergillus niger A2R8T9 MAE1 transport protein (SEQ ID NO: 1) are also useful in identifying and/or constructing other MAE1 transport proteins (and nucleic acids that encode them) suitable for use in accordance with the methods of the invention. In various embodiment, a suitable MAE1 transport protein for use in accordance with the methods of the invention has malonate transporter activity and comprises an amino acid sequence having a percentage identity to SEQ ID NO: 2 of at least 33%, for example, at least 50%, at least 55%, at least 60%, at least 65%, at least 70%, at least 75%, at least 80%, at least 85%, at least 90%, at least 92%, at least 94%, at least 95%, at least 96%, at least 97%, at least 98%, at least 99%, or 100% amino acid sequence identity to SEQ ID NO: 2. In other embodiments, a suitable MAE1 transport protein for use in accordance with the methods of the invention has malonate transporter activity and comprises an amino acid sequence having a percentage identity to SEQ ID NO: 3 of at least 50%, for example, at least 55%, at least 60%, at least 65%, at least 70%, at least 75%, at least 80%, at least 85%, at least 90%, at least 92%, at least 94%, at least 95%, at least 96%, at least 97%, at least 98%, at least 99%, or 100% amino acid sequence identity to SEQ ID NO: 3.

### MAE1 Transport Proteins Obtained from Schizosaccharomyces Species or Homologous Thereto

In addition to MAE1 transport proteins obtained from Aspergillus species and their homologous counterparts, the invention also provides MAE1 transport proteins obtained from Schizosaccharomyces species (and homologous counterpart proteins) suitable for use in accordance with the methods of the invention. In some embodiments, the recombinant host cell capable of producing malonate comprises a nucleic acid encoding a MAE1 transport protein obtained from a Schizosaccharomyces species that results in expression of the transport protein and provides an increase in the yield, titer, and/or productivity of malonate relative to a control cell that does not express the transport protein, or produces less of it. In various embodiments, the nucleic acid encoding a MAE1 transport protein is obtained from an organism selected from the group consisting of, but not limited to, Schizosaccharomyces cryophilus, Schizosaccharomyces japonica, Schizosaccharomyces octosporus, and Schizosaccharomyces pombe.

In one embodiment, the recombinant host cell capable of producing malonate comprises a nucleic acid encoding a Schizosaccharomyces pombe P50537 MAE1 transport protein (SEQ ID NO: 4) and provides an increase in the yield, titer, and/or productivity of malonate relative to a control cell that does not express the Schizosaccharomyces pombe P50537 MAE1 transport protein, or produces less of it. In another embodiment, the recombinant host cell capable of producing malonate comprises a nucleic acid encoding a Schizosaccharomyces pombe O59815 MAE1 transport protein (SEQ ID NO: 5) and provides an increase in the yield, titer, and/or productivity of malonate relative to a control cell that does not express the Schizosaccharomyces pombe O59815 MAE1 transport protein. Example 1 demonstrates how practice of the invention using Schizosaccharomyces pombe P50537 and O59815 MAE1 transport proteins (SEQ ID NOs: 4 and 5) in recombinant Pichia host cells increased malonate titer and yield relative to control cells not expressing these MAE1 transporters.

Suitable MAE1 transport proteins for use in accordance with the methods of the invention include those that are homologous to the Schizosaccharomyces pombe P50537 MAE1 transport protein sequence (SEQ ID NO: 4). In one embodiment, suitable MAE1 transport proteins for use in accordance with the methods of the invention have at least 33% identity to this MAE1 transport protein reference sequence. In another embodiment, suitable MAE1 transport proteins for use in the methods of invention have at least 50% identity to SEQ ID NO: 4. In various embodiments, the MAE1 transport protein has malonate transporter activity and comprises an amino acid sequence having at a percentage sequence identity to SEQ ID NO: 4 of at least 50%, for example, at least 55%, at least 60%, at least 65%, at least 70%, at least 75%, at least 80%, at least 85%, at least 90%, at least 92%, at least 94%, at least 95%, at least 96%, at least 97%, at least 98%, at least 99%, or 100% amino acid sequence identity to SEQ ID NO: 4. In some embodiments, a MAE1 transport protein with equal to or greater than 33% identity to the reference sequence SEQ ID NO: 4 is expressed in a recombinant host cell capable of producing malonate and is used to increase the production of malonate in said host cell relative the parental host cell.

The invention also provides expression vectors for expressing Schizosaccharomyces pombe O59815 MAE1 transport protein (SEQ ID NO: 5). The natural coding sequence can be used in identifying and/or constructing MAE1 transport protein coding sequences suitable for use in accordance with the methods of the invention. In various embodiments, a suitable MAE1 transport protein for use in accordance with the methods of the invention has malonate transporter activity and comprises an amino acid sequence having a percentage identity to SEQ ID NO: 5 of at least 50%, for example, at at least 55%, at least 60%, at least 65%, at least 70%, at least 75%, at least 80%, at least 85%, at least 90%, at least 92%, at least 94%, at least 95%, at least 96%, at least 97%, at least 98%, at least 99%, or 100% amino acid sequence identity to SEQ ID NO: 5. In some embodiments, a MAE1 transport protein with equal to or greater than 50% identity to the reference sequence SEQ ID NO: 5 is expressed in a recombinant host cell capable of producing malonate and provides an increase in malonate production relative to a parental cell not expressing said MAE1 transport protein.

### MAE1 Consensus Sequences

MAE1 transport proteins suitable for use in the compositions and methods of the invention include those MAE1 transport proteins homologous to MAE1 consensus sequences described herein. A consensus sequence provides a sequence of amino acids in which each position identifies the amino acid (if a specific amino acid is identified) or a subset of amino acids (if a position is identified as variable) most likely to be found at a specified position in a MAE1 transport protein. At positions in a consensus sequence where one of a subset of amino acids can be present, the following abbreviations are used below when referring to subsets of amino acids: B represents that one of the amino acids R, K, or H is present at the indicated position; J represents that one of the amino acids D or E is present at the indicated position; O represents that one of the amino acids I, L, or V is present at the indicated position. The percent sequence identity of a protein relative to a consensus sequence is determined by aligning the query protein sequence against the consensus sequence.

Proteins homologous to MAE1 consensus sequences have the following characteristics: (1) is capable of transporter activity that is identical, or essentially identical, or at least substantially similar with respect to ability to transport malonate across the cell membrane to that of one of the MAE1 transport proteins exemplified herein; (2) it shares substantial sequence identity with a MAE1 consensus sequence described herein; and/or (3) it possesses a substantial number of highly conserved amino acids found in a MAE1 consensus sequence described herein.

Two MAE1 consensus sequences provided herein are useful in identifying and constructing nucleic acids that encode MAE1 proteins suitable for use in the methods of the invention: (i) an MAE1 consensus sequence based on Aspergillus MAE1 transport proteins and referred to herein as an “Aspergillus MAE1 consensus sequence” (SEQ ID NO: 7); and (ii), a MAE1 consensus sequence based on both Aspergillus and Schizosaccharomyces pombe MAE1 transport proteins (SEQ ID NO: 8).

In various embodiments, a recombinant host cell capable of producing malonate expresses an MAE1 transport protein with at least 45% sequence identity to SEQ ID NO: 7 and provides an increase in malonate yield, titer, and/or productivity relative to a control cell that does not express said MAE1 transport protein. In some embodiments, the recombinant host cell expresses a protein with at least 80% identity to SEQ ID NO: 7. In still further embodiments, the recombinant host cell expresses a protein with a least 85%, at least 90%, at least 95%, or greater than 95% sequence identity to SEQ ID NO: 7. For example, the Aspergillus niger A2R8T9 (SEQ ID NO: 1), Aspergillus kawachi G7XR17 (SEQ ID NO: 2), Aspergillus terreus Q0D1U9 (SEQ ID NO: 3), Schizosaccharomyces pombe P50537 (SEQ ID NO: 4), and Schizosaccharomyces pombe O59815 (SEQ ID NO: 5) sequences are 100%, 100%, 94%, 52%, and 49% identical to the Aspergillus MAE1 consensus sequence (SEQ ID NO: 7); therefore, all five of these sequence are homologous to consensus sequence SEQ ID NO: 7. Additional proteins homologous to consensus sequence SEQ ID NO: 7 include, but are not limited to, those selected from the group consisting of: UniProt ID Q0D1U9 (94% identity to Seq ID NO: 7), UniProt ID B8N8E0 (89% identity to Seq ID NO: 7), UniProt ID I7ZSL4 (89% identity to Seq ID NO: 7), UniProt ID S8AYC2 (88% identity to Seq ID NO: 7), UniProt ID A1C406 (87% identity to Seq ID NO: 7), UniProt ID Q2UHT6 (87% identity to Seq ID NO: 7), UniProt ID A1DB74 (86% identity to Seq ID NO: 7), UniProt ID K9GWN1 (86% identity to Seq ID NO: 7), UniProt ID K9GI69 (86% identity to Seq ID NO: 7), UniProt ID Q5BDA8 (86% identity to Seq ID NO: 7), UniProt ID W6Q7W6 (86% identity to Seq ID NO: 7), UniProt ID B6HF90 (85% identity to Seq ID NO: 7), UniProt ID V5I2J6 (85% identity to Seq ID NO: 7), UniProt ID B0YA01 (84% identity to Seq ID NO: 7), and UniProt ID Q4WCF3 (84% identity to Seq ID NO: 7); any of these proteins are suitable for expression in recombinant host cells capable of producing malonate in order to provide an increase in malonate yield, titer, and/or productivity.

A number of amino acids in consensus sequence SEQ ID NO: 7 are highly conserved, and a majority of these amino acids, often more than 90%, and sometimes all of these amino acids are found in MAE1 transport proteins homologous to consensus sequence SEQ ID NO: 7. There are 279 highly conserved amino acids in SEQ ID NO: 7; namely: T5, P8, G9, S10, S11, S13, D14, O40, P49, G50, V52, G53, R55, E56, R57, O58, R59, H60, F61, T62, W63, A64, W65, Y66, T67, L68, T69, M70, S71, G73, G74, L75, A76, L77, L78, O79, Q82, P83, F86, G88, L89, B90, J91, 192, V96, Y97, L99, N100, O101, F103, F104, O106, V107, U109, M111, A112, R114, F115, I116, L117, H118, J123, S124, L125, H127, J128, R129, E130, G131, O132, F133, F134, P135, T136, F137, W138, L139, S140, I141, A142, T143, I145, T146, G147, L148, Y149, B150, F152, G153, J154, D155, F160, L164, L167, F168, W169, I170, Y171, C172, T175, O177, A179, V180, Q182, Y183, S184, O186, F187, K191, Y192, L194, T196, M198, P199, W201, I202, L203, P204, A205, F206, P207, V208, M209, L210, S211, G212, T213, I214, A215, S216, V217, I218, Q222, P223, A224, I228, P229, O231, O232, A233, G234, T236, F237, Q238, G239, L240, G241, F242, S243, I244, S245, M248, Y249, A250, H251, Y252, O253, G254, R255, L256, M257, E258, G260, L261, P262, E265, H266, R267, P268, G269, M270, F271, 1272, V274, G275, P276, P277, A278, F279, T280, A281, L282, A283, L284, V285, G286, M287, K289, L291, P292, D294, F295, Q296, O297, O298, G299, D300, A303, D306, R308, O309, L313, A314, O315, O319, F320, L321, W322, A323, L324, 5325, W327, F328, F329, C330, 1331, A332, O334, A335, V336, V337, R338, S339, P340, P341, F344, H345, L346, W348, A350, M351, V352, F353, P354, N355, T356, G357, F358, T359, L360, A361, T362, I363, L365, S371, G373, O374, G376, V377, T379, A380, M381, S382, O383, O₃₈₅, O386, F389, O390, F391, V392, O394, S395, O397, R398, A399, V400, I401, R402, K403, D404, I405, M406, P408, G409, D411, E412, D413, V414, and E416. In various embodiments, a recombinant host cell capable of producing malonate expresses an MAE1 transport protein having at least 50% of these highly conserved amino acids, and wherein said host cell produces an increased amount (yield, titer, and/or productivity) of malonate as compared to a control cell that does not express said MAE1 transport protein. For example, Schizosaccharomyces pombe P50537 (SEQ ID NO: 4) and Schizosaccharomyces pombe O59815 (SEQ ID NO: 5) have 72% and 66% of these highly conserved amino acids, respectively, and thus have a substantial number of these highly conserved amino acids. In some embodiments, a recombinant host cell capable of producing malonate expresses an MAE1 transport protein having at least 90% of these highly conserved amino acids, and wherein said host cell produces an increased amount (yield, titer, and/or productivity) of malonate as compared to a control cell that does not express said MAE1 transport protein. MAE1 transport proteins A2R8T9 (SEQ ID NO: 1) and G7XR17 (SEQ ID NO: 2) both have 100% of these highly conserved amino acids, and MAE1 transport protein Q0D1U9 (SEQ ID NO: 3) has 94% of these highly conserved amino acids; therefore, these proteins also possess a substantial number of these highly conserved amino acids.

In addition to the Aspergillus MAE1 consensus sequence, a MAE1 consensus sequence based on both Aspergillus and Schizosaccharomyces pombe MAE1 transport proteins (SEQ ID NO: 8) is also useful for identifying and constructing nucleic acids that encode MAE1 transport proteins suitable for use in accordance with the compositions and methods of the invention. In various embodiments, a recombinant host cell capable of producing malonate expresses an MAE1 transport protein with at least 80% sequence identity to SEQ ID NO: 8 and provides an increase in malonate yield, titer, and/or productivity relative to a control cell that does not express said MAE1 transport protein. In other embodiments, the recombinant host cell expresses a protein with a least 90%, at least 95%, or greater than 95% sequence identity to SEQ ID NO: 8. For example, the Aspergillus niger A2R8T9 (SEQ ID NO: 1), Aspergillus kawachi G7XR17 (SEQ ID NO: 2), Aspergillus terreus Q0D1U9 (SEQ ID NO: 3), Schizosaccharomyces pombe P50537 (SEQ ID NO: 4), and Schizosaccharomyces pombe O59815 (SEQ ID NO: 5) sequences are 95%, 95%, 98%, 85%, and 84% identical to MAE1 consensus sequence SEQ ID NO: 8, respectively; therefore, all five of these sequence are homologous to consensus sequence SEQ ID NO: 8.

A number of amino acids in consensus sequence SEQ ID NO: 8 are highly conserved, and a majority of these amino acids, often more than 90%, and sometimes all of these amino acids are found in MAE1 transport proteins homologous to consensus sequence SEQ ID NO: 8. There are 118 highly conserved amino acids in SEQ ID NO: 8; namely: O23, R40, O41, H43, F44, T45, W46, W48, M53, G56, G57, O58, O61, F69, G71, L72, O75, O79, O84, O89, R97, F98, I99, U106, E112, O114, F115, T118, L121, I123, T125, I127, L130, O146, L149, I152, O159, O162, O167, F168, O175, T177, P180, O183, L184, P185, F187, P188, M190, O191, G193, I195, A196, O199, Q203, P204, A205, O212, O213, G215, F218, Q219, G220, L221, G222, F223, O225, A231, R236, G241, L242, R248, P249, G250, M251, F252, V255, P257, P258, F260, U261, L263, L265, O266, O279, G280, O294, O300, F301, L305, C311, O312, A313, O315, O318, F325, W329, A331, O333, F334, N336, G338, O346, S352, O364, O366, O371, O373, O378, R379, A380, J385, O386, P389, G390, D392, E393, and D394. In various embodiments, a recombinant host cell capable of producing malonate expresses an MAE1 transport protein having at least 95% of these highly conserved amino acids, and wherein said host cell produces an increased amount (yield, titer, and/or productivity) as compared to a control cell that does not express said MAE1 transport protein. In some embodiments, a recombinant host cell capable of producing malonate expresses an MAE1 transport protein having all of these highly conserved amino acids, and wherein said host cell produces an increased amount (yield, titer, and/or productivity) as compared to a control cell that does not express said MAE1 transport protein. For example, 100% of the highly conserved amino acids in consensus sequence SEQ ID NO: 8 are found in MAE1 transport proteins encoded by SEQ ID NOs: 1, 2, 3, 4, and 5; thus, all five of these proteins have a substantial number of the highly conserved amino acids in SEQ ID NO: 8.

### Additional Sources of MAE1 Transport Proteins

As described above, nucleic acids encoding MAE1 transport proteins suitable for use in accordance with the methods of the invention may be obtained from organisms other than Aspergillus and Schizosaccharomyces species. In various embodiments, the recombinant host cell capable of producing malonate comprises a nucleic acid encoding a MAE1 transport protein obtained from an organism selected from the group consisting of, but not limited to, Ajellomyces capsulatus, Arthrobotrys oligospora, Arthroderma benhamiae, Arthroderma gypseum, Arthroderma otae, Baudoinia compniacensis, Beauveria bassiana, Bipolaris oryzae, Bipolaris victoriae, Bipolaris zeicola, Blumeria graminis, Botryosphaeria parva, Botryotinia fuckeliana, Byssochlamys spectabilis, Capronia coronata, Capronia epimyces, Chaetomium globosum, Chaetomium thermophilum, Cladophialophora carrionii, Cladophialophora psammophila, Cladophialophora yegresii, Claviceps purpurea, Coccidioides immitis, Coccidioides posadasii, Cochliobolus heterostrophus, Cochliobolus sativus, Colletotrichum gloeosporioides, Colletotrichum graminicola, Colletotrichum higginsianum, Colletotrichum orbiculare, Coniosporium apollinis, Cordyceps militaris, Cyphellophora europaea, Dactylellina haptotyla, Emericella nidulans, Eutypa lata, Exophiala dermatitidis, Fusarium oxysporum, Fusarium pseudograminearum, Gaeumannomyces graminis, Gibberella fujikuroi, Gibberella moniliformis, Gibberella zeae, Glarea lozoyensis, Grosmannia clavigera, Hypocrea atroviridis, Hypocrea jecorina, Hypocrea virens, Leptosphaeria maculans, Macrophomina phaseolina, Magnaporthe oryzae, Magnaporthe poae, Malassezia sympodialis, Marssonina brunnea, Metarhizium acridum, Metarhizium anisopliae, Mycosphaerella fijiensis, Mycosphaerella graminicola, Mycosphaerella pini, Nectria haematococca, Neosartorya fischeri, Neosartorya fumigata, Neurospora crassa, Neurospora tetrasperma, Ophiocordyceps sinensis, Penicillium chrysogenum, Penicillium digitatum, Penicillium oxalicum, Penicillium roqueforti, Pestalotiopsis fici, Phaeosphaeria nodorum, Podospora anserina, Pyrenophora teres, Pyrenophora tritici-repentis, Saccharomyces cerevisiae, Sclerotinia borealis, Sclerotinia sclerotiorum, Setosphaeria turcica, Sordaria macrospora, Sphaerulina musiva, Thielavia heterothallica, Thielavia terrestris, Togninia minima, Trichophyton equinum, Trichophyton rubrum, Trichophyton tonsurans, Trichophyton verrucosum, Verticillium alfalfae, and Verticillium dahlia.

### Expression Vectors

In a second aspect, the invention provides recombinant expression vectors encoding one or more MAE1 transport protein(s) that results in expression of the transport protein and provides an increase in the yield, titer, and/or productivity of malonate relative to a control cell that does not express the transport protein, or produces less of it. In various embodiments of the invention, the recombinant host cell has been modified by “genetic engineering” to produce a recombinant MAEI transport protein and secrete malonate. The host cell is typically engineered via recombinant DNA technology to express heterologous nucleic acids that encode a MAE1 transport protein, which is either a mutated version of a naturally occurring MAE1 transport protein, or a non-naturally occurring MAE1 transport protein prepared in accordance with one of the reference sequences provided herein, or is a naturally occurring MAE1 transport protein with MAE1 transport protein activity that is either overexpressed in the host cell in which it naturally occurs or is heteroloccously expressed in a host cell in which it does not naturally occur.

Nucleic acid constructs of the present invention include expression vectors that comprise nucleic acids encoding one or more MAE1 transport proteins. The nucleic acids encoding the proteins are operably linked to promoters and optionally other control sequences such that the subject proteins are expressed in a host cell containing the expression vector when cultured under suitable conditions. The promoters and control sequences employed depend on the host cell selected for the production of malonate. Thus, the invention provides not only expression vectors but also nucleic acid constructs useful in the construction of expression vectors. Methods for designing and making nucleic acid constructs and expression vectors generally are well known to those skilled in the art and so are only briefly reviewed herein.

Nucleic acids encoding the MAE1 transport protein can be prepared by any suitable method known to those of ordinary skill in the art, including, for example, direct chemical synthesis and cloning. Further, nucleic acid sequences for use in the invention can be obtained from commercial vendors that provide de novo synthesis of the nucleic acids.

A nucleic acid encoding the desired protein can be incorporated into an expression vector by known methods that include, for example, the use of restriction enzymes to cleave specific sites in an expression vector, e.g., plasmid, thereby producing an expression vector of the invention. Some restriction enzymes produce single stranded ends that may be annealed to a nucleic acid sequence having, or synthesized to have, a terminus with a sequence complementary to the ends of the cleaved expression vector. The ends are then covalently linked using an appropriate enzyme, e.g., DNA ligase. DNA linkers may be used to facilitate linking of nucleic acids sequences into an expression vector.

A set of individual nucleic acid sequences can also be combined by utilizing polymerase chain reaction (PCR)-based methods known to those of skill in the art. For example, each of the desired nucleic acid sequences can be initially generated in a separate PCR. Thereafter, specific primers are designed such that the ends of the PCR products contain complementary sequences. When the PCR products are mixed, denatured, and reannealed, the strands having the matching sequences at their 3′ ends overlap and can act as primers for each other. Extension of this overlap by DNA polymerase produces a molecule in which the original sequences are “spliced” together. In this way, a series of individual nucleic acid sequences may be joined and subsequently transduced into a host cell simultaneously. Thus, expression of each of the plurality of nucleic acid sequences is effected.

A typical expression vector contains the desired nucleic acid sequence preceded and optionally followed by one or more control sequences or regulatory regions, including a promoter and, when the gene product is a protein, ribosome binding site, e.g., a nucleotide sequence that is generally 3-9 nucleotides in length and generally located 3-11 nucleotides upstream of the initiation codon that precede the coding sequence, which is followed by a transcription terminator in the case of E. coli or other prokaryotic hosts. See Shine et al., Nature 254:34 (1975) and Steitz, in Biological Regulation and Development: Gene Expression (ed. R. F. Goldberger), vol. 1, p. 349 (1979) Plenum Publishing, N.Y. In the case of eukaryotic hosts like yeast a typical expression vector contains the desired nucleic acid coding sequence preceded by one or more regulatory regions, along with a Kozak sequence to initiate translation and followed by a terminator. See Kozak, Nature 308:241-246 (1984).

Regulatory regions or control sequences include, for example, those regions that contain a promoter and an operator. A promoter is operably linked to the desired nucleic acid coding sequence, thereby initiating transcription of the nucleic acid sequence via an RNA polymerase. An operator is a sequence of nucleic acids adjacent to the promoter, which contains a protein-binding domain where a transcription factor can bind. Transcription factors activate or repress transcription initiation from a promoter. In this way, control of transcription is accomplished, based upon the particular regulatory regions used and the presence or absence of the corresponding transcription factor. Non-limiting examples for prokaryotic expression include lactose promoters (LacI repressor protein changes conformation when contacted with lactose, thereby preventing the Lad repressor protein from binding to the operator) and tryptophan promoters (when complexed with tryptophan, TrpR repressor protein has a conformation that binds the operator; in the absence of tryptophan, the TrpR repressor protein has a conformation that does not bind to the operator). Non-limiting examples of promoters to use for eukaryotic expression include pACH11, pACO11, pADH1, pADH2, pALD4, pCIT1, pCUP1, pENO2, pFBA1, pGAL1, pGAPD, pHSP15, pHXK21, pHXT7, pJEN11, pMDH21, pMET3, pPDC1, pPGI1, pPGK1, pPHO5, pPOX11, pPRB1, pPYK1, pREV1, pRNR2, pRPL1, pSCT1, pSDH1, pTDH2, pTDH3, pTEF1, pTEF2, pTPI1, and pTPI11. As will be appreciated by those of ordinary skill in the art, a variety of expression vectors and components thereof may be used in the present invention.

Although any suitable expression vector may be used to incorporate the desired sequences, readily available expression vectors include, without limitation: plasmids, such as pESC, pTEF, p414CYC1, p414GALS, pSC101, pBR322, pBBR1MCS-3, pUR, pEX, pMR100, pCR4, pBAD24, pUC19, pRS series; and bacteriophages, such as M13 phage and λ phage. Of course, such expression vectors may only be suitable for particular host cells or for expression of particular MAE1 transport proteins. One of ordinary skill in the art, however, can readily determine through routine experimentation whether any particular expression vector is suited for any given host cell or protein. For example, the expression vector can be introduced into the host cell, which is then monitored for viability and expression of the sequences contained in the vector. In addition, reference may be made to the relevant texts and literature, which describe expression vectors and their suitability to any particular host cell. In addition to the use of expression vectors, strains are built where expression cassettes are directly integrated into the host genome.

The expression vectors are introduced or transferred, e.g. by transduction, transfection, or transformation, into the host cell. Such methods for introducing expression vectors into host cells are well known to those of ordinary skill in the art. For example, one method for transforming S. cerevisiae with an expression vector involves a lithium acetate/polyethylene glycol treatment wherein the expression vector is introduced into the host cell following treatment with a solution comprising lithium acetate and polyethylene glycol.

For identifying whether a nucleic acid has been successfully introduced or into a host cell, a variety of methods are available. For example, a culture of potentially transformed host cells may be separated, using a suitable dilution, into individual cells and thereafter individually grown and tested for expression of a desired gene product of a gene contained in the introduced nucleic acid. For example, an often-used practice involves the selection of cells based upon antibiotic resistance that has been conferred by antibiotic resistance-conferring genes in the expression vector, such as the beta lactamase (amp), aminoglycoside phosphotransferase (neo), and hygromycin phosphotransferase (hyg, hph, hpt) genes.

Typically, a host cell of the invention will have been transformed with at least one expression vector. Once the host cell has been transformed with the expression vector, the host cell is cultured in a suitable medium containing a carbon source, such as a sugar (e.g., glucose). As the host cell is cultured, expression of the enzyme for producing malonate and secretion of malonate into the fermentation broth occurs.

If a host cell of the invention is to include more than one heterologous gene, then multiple genes can be expressed from one or more vectors. For example, a single expression vector can comprise one, two, or more genes encoding one, two, or more MAE1 transport protein(s) and/or other proteins providing some useful function, e.g. producing malonate. The heterologous genes can be contained in a vector replicated episomally or in a vector integrated into the host cell genome, and where more than one vector is employed, then all vectors may replicate episomally (extrachromasomally), or all vectors may integrate, or some may integrate and some may replicate episomally. Chromosomal integration is typically used for cells that will undergo sustained propagation, e.g., cells used for production of malonate for industrial applications. While a “gene” is generally composed of a single promoter and a single coding sequence, in certain host cells, two or more coding sequences may be controlled by one promoter in an operon. In some embodiments, a two or three operon system is used.

In some embodiments, the coding sequences employed have been modified, relative to some reference sequence, to reflect the codon preference of a selected host cell. Codon usage tables for numerous organisms are readily available and are used to guide sequence design. The use of prevalent codons of a given host organism generally improves translation of the target sequence in the host cell. As one non-limiting example, in some embodiments the subject nucleic acid sequences will be modified for yeast codon preference (see, for example, Bennetzen et al., J. Biol. Chem. 257: 3026-3031 (1982)). In some embodiments, the nucleotide sequences are modified to include codons optimized for S. cerevisiae codon preference.

Nucleic acids can be prepared by a variety of routine recombinant techniques. Briefly, the subject nucleic acids can be prepared from genomic DNA fragments, cDNAs, and RNAs, all of which can be extracted directly from a cell or recombinantly produced by various amplification processes including but not limited to PCR and rt-PCR. Subject nucleic acids can also be prepared by a direct chemical synthesis.

The nucleic acid transcription levels in a host microorganism can be increased (or decreased) using numerous techniques. For example, the copy number of the nucleic acid can be increased through use of higher copy number expression vectors comprising the nucleic acid sequence, or through integration of multiple copies of the desired nucleic acid into the host microorganism's genome. Non-limiting examples of integrating a desired nucleic acid sequence onto the host chromosome include recA-mediated recombination, lambda phage recombinase-mediated recombination and transposon insertion. Nucleic acid transcript levels can be increased by changing the order of the coding regions on a polycistronic mRNA or breaking up a polycistronic operon into multiple poly- or mono-cistronic operons each with its own promoter. RNA levels can be increased (or decreased) by increasing (or decreasing) the strength of the promoter to which the protein-coding region is operably linked.

The translation level of a desired polypeptide sequence in a host microorganism can also be increased in a number of ways. Non-limiting examples include increasing the mRNA stability, modifying the ribosome binding site (or Kozak) sequence, modifying the distance or sequence between the ribosome binding site (or Kozak sequence) and the start codon of the nucleic acid sequence coding for the desired polypeptide, modifying the intercistronic region located 5′ to the start codon of the nucleic acid sequence coding for the desired polypeptide, stabilizing the 3′-end of the mRNA transcript, modifying the codon usage of the polypeptide, altering expression of low-use/rare codon tRNAs used in the biosynthesis of the polypeptide. Determination of preferred codons and low-use/rare codon tRNAs can be based on a sequence analysis of genes obtained from the host microorganism.

The polypeptide half-life, or stability, can be increased through mutation of the nucleic acid sequence coding for the desired polypeptide, resulting in modification of the desired polypeptide sequence relative to the control polypeptide sequence. When the modified polypeptide is an enzyme, the activity of the enzyme in a host may be altered due to increased solubility in the host cell, improved function at the desired pH, removal of a domain inhibiting enzyme activity, improved kinetic parameters (lower Km or higher Kcat values) for the desired substrate, removal of allosteric regulation by an intracellular metabolite, and the like. Altered/modified enzymes can also be isolated through random mutagenesis of an enzyme, such that the altered/modified enzyme can be expressed from an episomal vector or from a recombinant gene integrated into the genome of a host microorganism.

### Methods for Producing Malonate

In a third aspect, the invention provides a method for the production of malonate, the method comprising the steps of: (a) culturing a population of any of the recombinant host cells described herein in a fermentation broth with a carbon source under conditions suitable for making malonate; and (b) recovering said malonate from the fermentation broth.

In various embodiments, a recombinant host cell capable of producing malonate and comprising a heterologous nucleic acid encoding a MAE1 transport protein provides an increased yield, titer, and/or productivity of malonate compared to a parent cell not comprising the heterologous nucleic acid encoding the MAE1 transport protein, but is otherwise genetically identical. In some embodiments, the increased amount is at least 5%, 10%, 15%, 20%, 25%, 30%, 35%, 40%, 45%, 50%, 55%, 60%, 65%, 70%, 75%, 80%, 85%, 90%, 95%, 100% or greater than 100%, as measured, for example, in yield, titer, productivity, on a per unit volume of cell culture basis, on a per unit dry cell weight basis, on a per unit volume of cell culture per unit time basis, or on a per unit dry cell weight per unit time basis.

In some embodiments, the host cell produces an elevated level of malonate that is at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 1.25-fold, at least about 1-fold, at least about 2-fold, at least about 4-fold, or more, higher than the level of malonate produced by a parent cell, on a per unit volume of cell culture basis. For example, as described in Example 1, strain LPK15010 comprising Aspergillus niger A2R8T9 MAE1 transport protein produced greater than 2-fold more malonate than the parental control strain, LPK15003 (i.e., 36±5.6 mM in LPK15003 versus 75±10.3 mM in LPK15010).

In some embodiments, the host cell produces an elevated level of malonate that is at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 1.25-fold, at least about 2-fold, at least about 4-fold, at least about 10-fold, or more, higher than the level of malonate produced by the parent cell, on a per unit volume of cell culture per unit time basis.

Materials and methods for the maintenance and growth of microbial cultures are well known to those skilled in the art of microbiology or fermentation science (see, for example, Bailey et al., Biochemical Engineering Fundamentals, second edition, McGraw Hill, New York, 1986). Consideration must be given to appropriate culture medium, pH, temperature, and requirements for aerobic, microaerobic, or anaerobic conditions, depending on the specific requirements of the host cell, the fermentation, and the process.

The methods of producing malonate provided herein may be performed in a suitable culture medium, in a suitable container, including but not limited to a cell culture plate, a flask, or a fermenter. Further, the methods can be performed at any scale of fermentation known in the art to support industrial production of microbial products. Any suitable fermenter may be used including a stirred tank fermenter, an airlift fermenter, a bubble fermenter, or any combination thereof.

In some embodiments, the culture medium is any culture medium in which a genetically modified microorganism capable of producing malonate can subsist. In some embodiments, the culture medium is an aqueous medium comprising assimilable carbon, nitrogen and phosphate sources. Such a medium can also include appropriate salts, minerals, metals and other nutrients. In some embodiments, the carbon source and each of the essential cell nutrients are added incrementally or continuously to the fermentation media, and each required nutrient is maintained at essentially the minimum level needed for efficient assimilation by growing cells.

The invention, having been described in detail, is illustrated by the following examples, which should not be construed as limiting the invention, given its diverse aspects, embodiments, and applications.

### EXAMPLES

### Example 1

### Increasing Malonate Titer in Recombinant Pichia kudriavzevii Through Heterologous Expression of MAE1 Transport Proteins

In this example, recombinant P. kudriavzevii host cells capable of producing malonate were transformed with nucleic acids encoding MAE1 transport proteins and were shown to increase malonate production.

Recombinant P. kudriavzevii LPK15001 was used as the base strain, and harbors a single copy of a malonyl-CoA hydrolase at the GPD1 locus. The following strains were constructed by chromosomal integration at the FAS1 locus with nucleic acids encoding the indicated protein, LPK15003: F6A-4; LPK15004: F6A-4 and A1C406; LPK15005: F6A-4 and G7XR17; LPK15006: F6A-4 and O59815; LPK15009: F6A-4 and P50537; LPK15010: F6A-4 and A2R8T9; and LPK15011: F6A-4 and Q0DIU9. Protein F6A-4 is a malonyl-CoA hydrolase (UniProt ID F6AA82 containing point mutations E95N/F304R/Q348A); thus, strain LPK15003 is the control strain establishing the baseline level of malonate production in the absence of heterologous expression of a MAE1 transport protein. In all strains, the pTDH1 promoter was used to control transcription of the gene encoding the F6A-4 malonyl-CoA hydrolase. The pPGK1 promoter was used to control transcription of the genes encoding the G7XR17, O59815, P50537, A2R8T9, and Q0DIU9 proteins. TEF1 terminators were inserted behind all heterologous genes described above to stop transcription. A kanamycin resistance marker was included in the assembled nucleic acid to enable selection for positive integrants. The 5′ and 3′ ends of the nucleic acid contained between 962 and 976 basepairs of DNA sequence homologous to the P. kudriavzevii FAS1 gene were included to target nucleic acid insertion into the FAS1 locus of the host genome.

Nucleic acids were transformed into P. kudriavzevii LPK15001 using a lithium acetate/PEG protocol. In brief, a colony LPK15001 was inoculated into 50 mL of YNB yeast medium in a culture flask, and incubated at 30° C. and 85% relative humidity with shaking (200 rpm) for approximately 4 hours. The culture was then placed on ice for approximately 15 minutes, centrifuged (×6000 g, 1 min), the supernatant removed, and the pellet resuspended in 50 ml of ice-cold, sterile water. The cells were then resuspended in approximately 3 ml of ice cold, sterile water and centrifuged (×6000 g, 1 min). The resulting pellet was resuspended in 500 μl of 30% glycerol, 0.1M lithium acetate at 4° C. The resuspended cells were then aliquoted into 50 μl volumes to which 5 μl ssDNA (salmon sperm ssDNA, 10 mg/ml), 145 μl 50% PEG (MW-6,500), and approximately 20 μl of the heterologous nucleic acid(s) encoding the expression cassettes were added. The mixture was incubated for 30 minutes at 30° C. and then for 45 minutes at 42° C. The transformations were then plated on YNB plates containing G418 antibiotic (500 μg/ml) to select for the presence of the kanamycin resistance cassette.

For production assays, individual colonies were next inoculated into 500 YNB growth medium supplemented with 8% w/v glucose. All cultures were inoculated into 2.2-ml volume 96-well plates. The culture plates were then incubated at 30° C. with shaking (250 rpm) for 5 days, at which point the fermentation broth was sampled.

Samples were centrifuged (x6000 g, 1 min) and the supernatant analyzed for malonate concentration. The separation of malonate was conducted on a Shimadzu Prominence XR HPLC connected to a refractive index detector and UV detector monitoring 210 nm. Product separation was performed on a Bio-Rad Aminex HPX-87h Fermentation Monitoring column. The UPLC was programmed to run isocratically using 5 mM H₂SO₄ as the eluent with a flow rate of 800 μL per minute. 10 μl were injected per sample, and the sample plate temperature was held at 4° C. Malonate standards began eluting at ˜8.0 minutes. Malonate concentrations (mM) in the fermentation broth were calculated by comparison to a standard curve prepared from authentic malonate prepared in water.

The results of this production assay were as follows: LPK15003: 36±5.6 mM, LPK15004: 36±5.4 mM, LPK15005: 76±10.6 mM, LPK15006: 50±7.6 mM, LPK15009: 73±7.0 mM, LPK15010: 75±10.3 mM, and LPK15011: 68±14.4 mM. Thus, strains LPK15006 and LPK15009, which expressed the Schizosaccharomyces pombe MAE1 transport proteins, provided approximately 1.4-fold and 2.0-fold increases in malonate production, respectively, relative to the LPK15003 control strain. Strains LPK15005, LPK15010, and LPK15011, which expressed the MAE1 transport proteins obtained from Aspergillus species, provided approximately 2.1-fold, 2.1-fold, and 1.9-fold increases in malonate production, respectively, relative to the LPK15003 control strain.

Upon sequencing of the transformed heterologous nucleic acid in strain LPK15004 a nucleotide deletion resulting in a frameshift mutation during translation was identified. Thus, strain LPK15004 in this example did not express the A1C406 MAE1 transport protein.

This example demonstrates, in accordance with the invention, that heterologous expression of nucleic acids encoding a wide variety of MAE1 transport proteins (i.e. A2R8T9, G7XR17, QOD1U9, P50537, and O59815) increased malonate production in recombinant yeast cells capable of producing malonate. Moreover, this example provides a readily conducted, efficient method to determine if a putative MAE1 transport protein is an MAE1 transport protein and efficiently secretes malonate into the fermentation broth.

### Example 2

### Increasing Malonate Productivity in Recombinant Pichia kudriavzevii Through Heterologous Expression of MAE1 Transport Proteins

In this example, yeast strains LPK15004 and LPK15010 (see Example 1 for strain construction details) is grown in fed-batch control in a 0.5 L bioreactor (Infors, Sixfors system). A single colony of LPK15004 is isolated from a YPD plate and cultured in 6 mL of YNB4 2% media (20 g/L glucose, 6.7 g/L YNB without amino acids (Sigma), and 150 mM succinic acid buffer pH 4.0). The culture is maintained at 30° C. for 24 hours, shaking at 200 rpm. The 6 mL of culture is combined with 4 mL of 50% (v/v) glycerol and aliquoted in 1 mL volumes into cryo-vials. Cyro-vials are frozen and maintained at −80c. One vial is used to inoculate 50 mL of fresh YNB4 2% media in a 250 mL baffled flask and grown for 24 hrs at 30° C., 200 rpm. This culture is used to inoculate 200 mL of YNB4 2% media with 0.1% antifoam. The fermentation is maintained at 30° C., at a pH of 5.0 maintained by the addition of ammonium hydroxide, potassium hydroxide, or sodium hydroxide. Oxygen transfer is controlled through two rushton impellers run at 1000 rpm, and using a sparger an air flow rate of 30 NL/hr using compressed air is maintained. The culture is grown overnight (˜20 h) to allow for glucose consumption prior to starting the fed-batch phase. The feed (150 g/L glucose, 13.4 g/L YNB without amino acids (Sigma)) is initiated automatically when the dissolved oxygen spikes sharply indicating depletion of glucose. Feed is delivered for 2 s, every 200 s. Samples are taken daily. Growth is monitored by measuring optical density at 600 nm (OD600). Concentration of glucose is measured using a glucose monitor (YSI Life Sciences). Production of malonic acid, acetic acid, succinic acid, and pyruvic acid is measured via HPLC as described in Example 1. Productivity is calculated as the malonate formation rate per unit volume over time, and is expressed as g/l/hr.

Strain LPK15010 provides a higher productivity relative to LPK15004, demonstrating that in accordance with the invention, that heterologous expression of the Aspergillus niger A2R8T9 MAE1 transport protein increases malonate productivity in recombinant yeast cells capable of producing malonate. Moreover, this example provides another readily conducted, efficient method to determine if a putative MAE1 transport protein is an MAE1 transport protein and efficiently secretes malonate into the fermentation broth.

### Example 3

### Increasing Malonate Productivity in Recombinant Pichia kudriavzevii Through Heterologous Expression of MAE1 Transport Proteins

In this example, recombinant P. kudriavzevii host cells capable of producing malonate were engineered to express a MAE1 transport protein resulting in increased malonate productivity relative to control cells that did not express an MAE1 transport protein.

The yeast used in this example was Pichia Kudriavzevii (ARS Culture Collection strain Y-134). Engineered yeast LPK3013 served as a control strain, and comprised two malonyl-CoA hydrolase expression cassettes: (i) one malonyl-CoA hydrolase expression cassette comprising a pTDH1 promoter, F6A-4 malonyl-CoA hydrolase (see Example 1 for a description of F6A-4), CYC1 terminator, and a hygromycin selection marker; this expression cassette was integrated at the GPD1 locus; and (ii) a second malonyl-CoA hydrolase expression cassette identical to the first with the exception that this cassette was integrated at the FAS1 locus and included a SUC2 selection marker in place of the hygromycin selection marker. Engineered strain LPK3020 was genetically identical to LPK3013 with the exception that LPK3020 was additionally engineered to express the Aspergillus kawachi G7XR17 MAE1 transport protein (SEQ ID NO: 2). The MAE1 transporter expression cassette comprised a pPGK1 promoter controlling expression of the Aspergillus kawachi G7XR17 MAE1 transport protein (SEQ ID NO: 2), and also included a TEF1 terminator cloned downstream of the gene; this expression cassette was integrated, along with the above-described malonyl-CoA hydrolase expression cassette, at the FAS1 locus using a kanamycin resistance marker. Thus, in this example, strain LPK3013 was the parental, control strain used to establish the baseline level of malonate productivity in the absence of expression of a MAE1 transport protein.

Nucleic acids were transformed into P. kudriavzevii strains using a lithium acetate/PEG protocol as described in Example 1. Single colonies of LPK30013 and LPK3020 were isolated from YPD solid media plates and cultured in 6 mL of YNB4 2% media (20 g/L glucose, 6.7 g/L YNB without amino acids (Sigma), and 150 mM succinic acid buffer pH 4.0). The culture was maintained at 30° C. for 24 hours, shaking at 200 rpm. The 6 mL of culture was combined with 4 mL of 50% (v/v) glycerol and aliquoted in 1 mL volumes into cryo-vials. Cyro-vials were frozen and maintained at −80° C. One vial of each strain was used to inoculate 50 mL of fresh YNB4 2% media in a 250 mL baffled flask and grown for 24 hrs at 30° C., 200 rpm. These cultures were used to inoculate 200 mL of YNB4 2% media with 0.1% antifoam. The fermentation was maintained at 30° C., and the pH was maintained at 5.0 by addition of ammonium hydroxide. Oxygen transfer was controlled through two rushton impellers run at 1000 rpm, and using a sparger an air flow rate of 30 NL/hr using compressed air was maintained. The culture was grown overnight (˜20 h) to allow for glucose consumption prior to starting the fed-batch phase. The feed (300 g/L glucose, 13.4 g/L Yeast Nitrogen Base without amino acids (Sigma)) was initiated automatically when the dissolved oxygen spiked sharply, indicating depletion of glucose. Feed was delivered for 2 s, every 200 s. Samples were taken daily. Growth was monitored by measuring optical density at 600 nm (OD600). Samples were centrifuged (×6000 g, 1 min) and the glucose and malonate concentrations in the supernatant analyzed. Glucose concentration was measured using a glucose monitor (YSI Life Sciences). The separation of malonate was conducted on a Shimadzu Prominence XR HPLC as described in Example 1.

Control strain LPK3013 provided a malonate productivity of 0.17 g/L/hr. Strain LPK3020, expressing the Aspergillus kawachi G7XR17 MAE1 transport protein, provided a malonate productivity of 0.424 g/L/hr. Thus, malonate productivity was increased nearly 2.5-fold in yeast expressing a MAE1 transport protein as compared to a parental, control strain that did not express said MAE1 transport protein.

This example demonstrates that in accordance with the invention, expression of an MAE1 transport protein in a host cell capable of producing malonate increases malonate productivity as compared to a host cell that do not express said MAE1 transport protein. Moreover, this example provides another readily conducted, efficient method to determine if a putative MAE1 transport protein is an MAE1 transport protein and efficiently secretes malonate into the fermentation broth.

1. A recombinant host cell capable of producing malonate wherein said host cell comprises a heterologous nucleic acid encoding a MAE1 transport protein and wherein said host cell produces an increased level of malonate relative to a parental host cell not comprising a heterologous nucleic acid encoding said MAE1 transport protein.

2. The host cell of claim 1, wherein said nucleic acid encoding a MAE1 transport protein is a nucleic acid encoding an MAE1 transport protein obtained from an Aspergillus species or is homologous thereto.

3. The host cell of claim 2, wherein said MAE1 transport protein is selected from the group consisting of SEQ ID NO: 1, SEQ ID NO: 2, and/or SEQ ID NO: 3.

4. The host cell of claim 2, wherein said MAE1 transport protein is Aspergillus niger A2R8T9 MAE1 transport protein (SEQ ID NO: 1).

5. The host cell of claim 1, wherein said nucleic acid encoding a MAE1 transport protein is a nucleic acid encoding a MAE1 transport protein obtained from a Schizosaccharomyces species or is homologous thereto.

6. The host cell of claim 5, wherein said MAE1 transport protein is SEQ ID NO: 4 or SEQ ID NO: 5.

7. The host cell of claim 1, wherein the MAE1 transport protein has at least 25% amino acid sequence identity to SEQ ID NO: 1.

8. The host cell of claim 1, wherein the MAE1 transport protein has at least 60% amino acid sequence identity to a MAE1 transport protein selected from the group consisting of SEQ IDs: 1, 2, or 3.

9. The host cell of claim 1, wherein the MAE1 transport protein has at least 45% amino acid sequence identity to consensus sequence SEQ ID NO: 7.

10. The host cell of claim 1, wherein the MAE1 transport protein has at least 80% amino acid sequence identity to consensus sequence SEQ ID NO: 8.

11. The host cell of claim 1, wherein said host cell that is a yeast host cell.

12. The host cell of claim 11, wherein said yeast host cell is selected from the group consisting of Pichia, Saccharomyces, and Yarrowia host cells.

13. The host cell of claim 11, wherein said yeast host cell is a Pichia kudriavzevii host cell.

14. A method for producing malonate, said method comprising fermenting a host cell of claim 1 under conditions such that malonate is produced and secreted into fermentation medium.


SEQUENCE LISTING (27 sequences)

SEQ ID NO: 1 - protein, 267 aa, Pseudomonas aeruginosa
MLNIVMIGCGAIGAGVLELLENDPQLRVDAVIVPRDSETQVRHRLASLRRPPRVLSALPAGERPDLLVECAGHRAIEQHVLPALAQGIPCLVVSVGALSEPGLVERLEAAAQAGGSRIELLPGAIGAIDALSAARVGGLESVRYTGRKPASAWLGTPGETVCDLQRLEKARVIFDGSAREAARLYPKNANVAATLSLAGLGLDRTQVRLIADPESCENVHQVEASGAFGGFELTLRGKPLAANPKTSALTVYSVVRALGNHAHAISI

SEQ ID NO: 2 - protein, 266 aa, Cupriavidius taiwanensis
MLHVSMVGCGAIGRGVLELLKSDPDVVFDVVIVPEHTMDEARGAVSALAPRARVATHLDDQRPDLLVECAGHHALEEHIVPALERGIPCMVVSVGALSEPGMAERLEAAARRGGTQVQLLSGAIGAIDALAAARVGGLDEVIYTGRKPARAWTGTPAEQLFDLEALTEATVIFEGTARDAARLYPKNANVAATVSLAGLGLDRTAVKLLADPHAVENVHHVEARGAFGGFELTMRGKPLAANPKTSALTVFSVVRALGNRAHAVSI

SEQ ID NO: 3 - protein, 540 aa, Tribolium castaneum
MPATGEDQDLVQDLIEEPATFSDAVLSSDEELFHQKCPKPAPIYSPISKPVSFESLPNRRLHEEFLRSSVDVLLQEAVFEGTNRKNRVLQWREPEELRRLMDFGVRGAPSTHEELLEVLKKVVTYSVKTGHPYFVNQLFSAVDPYGLVAQWATDALNPSVYTYEVSPVFVLMEEVVLREMRAIVGFEGGKGDGIFCPGGSIANGYAISCARYRFMPDIKKKGLHSLPRLVLFTSEDAHYSIKKLASFEGIGTDNVYLIRTDARGRMDVSHLVEEIERSLREGAAPFMVSATAGTTVIGAFDPIEKIADVCQKYKLWLHVDAAWGGGALVSAKHRHLLKGIERADSVTWNPHKLLTAPQQCSTLLLRHEGVLAEAHSTNAAYLFQKDKFYDTKYDTGDKHIQCGRRADVLKFWFMWKAKGTSGLEKHVDKVFENARFFTDCIKNREGFEMVIAEPEYTNICFWYVPKSLRGRKDEADYKDKLHKVAPRIKERMMKEGSMMVTYQAQKGHPNFFRIVFQNSGLDKADMVHFVEEIERLGSDL

SEQ ID NO: 4 - protein, 136 aa, Corynebacterium glutamicum
MLRTILGSKIHRATVTQADLDYVGSVTIDADLVHAAGLIEGEKVAIVDITNGARLETYVIVGDAGTGNICINGAAAHLINPGDLVIIMSYLQATDAEAKAYEPKIVHVDADNRIVALGNDLAEALPGSGLLTSRSI

SEQ ID NO: 5 - protein, 127 aa, Bacillus subtilis
MYRTMMSGKLHRATVTEANLNYVGSITIDEDLIDAVGMLPNEKVQIVNNNNGARLETYIIPGKRGSGVICLNGAAARLVQEGDKVIIISYKMMSDQEAASHEPKVAVLNDQNKIEQMLGNEPARTIL

SEQ ID NO: 6 - protein, 538 aa, Mannheimia succiniciproducens
MTDLNQLTQELGALGIHDVQEVVYNPSYELLFAEETKPGLEGYEKGTVTNQGAVAVNTGIFTGRSPKDKYIVLDDKTKDTVWWTSEKVKNDNKPMSQDTWNSLKGLVADQLSGKRLFVVDAFCGANKDTRLAVRVVTEVAWQAHFVTNMFIRPSAEELKGFKPDFVVMNGAKCTNPNWKEQGLNSENFVAFNITEGVQLIGGTWYGGEMKKGMFSMMNYFLPLRGIASMHCSANVGKDGDTAIFFGLSGTGKTTLSTDPKRQLIGDDEHGWDDEGVFNFEGGCYAKTINLSAENEPDIYGAIKRDALLENVVVLDNGDVDYADGSKTENTRVSYPIYHIQNIVKPVSKAGPATKVIFLSADAFGVLPPVSKLTPEQTKYYFLSGFTAKLAGTERGITEPTPTFSACFGAAFLSLHPTQYAEVLVKRMQESGAEAYLVNTGWNGTGKRISIKDTRGIIDAILDGSIDKAEMGSLPIFDFSIPKALPGVNPAILDPRDTYADKAQWEEKAQDLAGRFVKNFEKYTGTAEGQALVAAGPKA

SEQ ID NO: 7 - protein, 1193 aa, Aspergillus oryzae
MAAPFRQPEEAVDDTEFIDDHHEHLRDTVHHRLRANSSIMHFQKILVANRGEIPIRIFRTAHELSLQTVAIYSHEDRLSMHRQKADEAYMIGHRGQYTPVGAYLAGDEIIKIALEHGVQLIHPGYGFLSENADFARKVENAGIVFVGPTPDTIDSLGDKVSARRLAIKCEVPVVPGTEGPVERYEEVKAFTDTYGFPIIIKAAFGGGGRGMRVVRDQAELRDSFERATSEARSAFGNGTVFVERFLDKPKHIEVQLLGDSHGNVVHLFERDCSVQRRHQKVVEVAPAKDLPADVRDRILADAVKLAKSVNYRNAGTAEFLVDQQNRHYFIEINPRIQVEHTITEEITGIDIVAAQIQIAAGASLEQLGLTQDRISARGFAIQCRITTEDPAKGFSPDTGKIEVYRSAGGNGVRLDGGNGFAGAIITPHYDSMLVKCTCRGSTYEIARRKVVRALVEFRIRGVKTNIPFLTSLLSHPTFVDGNCWTTFIDDTPELFSLVGSQNRAQKLLAYLGDVAVNGSSIKGQIGEPKLKGDVIKPKLFDAEGKPLDVSAPCTKGWKQILDREGPAAFAKAVRANKGCLIMDTTWRDAHQSLLATRVRTIDLLNIAHETSYAYSNAYSLECWGGATFDVAMRFLYEDPWDRLRKMRKAVPNIPFQMLLRGANGVAYSSLPDNAIYHFCKQAKKCGVDIFRVFDALNDVDQLEVGIKAVHAAEGVVEATMCYSGDMLNPHKKYNLEYYMALVDKIVAMKPHILGIKDMAGVLKPQAARLLVGSIRQRYPDLPIHVHTHDSAGTGVASMIACAQAGADAVDAATDSMSGMTSQPSIGAILASLEGTEQDPGLNLAHVRAIDSYWAQLRLLYSPFEAGLTGPDPEVYEHEIPGGQLTNLIFQASQLGLGQQWAETKKAYEAANDLLGDIVKVTPTSKVVGDLAQFMVSNKLTPEDVVERAGELDFPGSVLEFLEGLMGQPFGGFPEPLRSRALRDRRKLEKRPGLYLEPLDLAKIKSQIREKFGAATEYDVASYAMYPKVFEDYKKFVQKFGDLSVLPTRYFLAKPEIGEEFHVELEKGKVLILKLLAIGPLSEQTGQREVFYEVNGEVRQVAVDDNKASVDNTSRPKADVGDSSQVGAPMSGVVVEIRVHDGLEVKKGDPLAVLSAMKMEMVISAPHSGKVSSLLVKEGDSVDGQDLVCKIVKA

SEQ ID NO: 8 - protein, 883 aa, Escherichia coli
MNEQYSALRSNVSMLGKVLGETIKDALGEHILERVETIRKLSKSSRAGNDANRQELLTTLQNLSNDELLPVARAFSQFLNLANTAEQYHSISPKGEAASNPEVIARTLRKLKNQPELSEDTIKKAVESLSLELVLTAHPTEITRRTLIHKMVEVNACLKQLDNKDIADYEHNQLMRRLRQLIAQSWHTDEIRKLRPSPVDEAKWGFAVVENSLWQGVPNYLRELNEQLEENLGYKLPVEFVPVRFTSWMGGDRDGNPNVTADITRHVLLLSRWKATDLFLKDIQVLVSELSMVEATPELLALVGEEGAAEPYRYLMKNLRSRLMATQAWLEARLKGEELPKPEGLLTQNEELWEPLYACYQSLQACGMGIIANGDLLDTLRRVKCFGVPLVRIDIRQESTRHTEALGELTRYLGIGDYESWSEADKQAFLIRELNSKRPLLPRNWQPSAETREVLDTCQVIAEAPQGSIAAYVISMAKTPSDVLAVHLLLKEAGIGFAMPVAPLFETLDDLNNANDVMTQLLNIDWYRGLIQGKQMVMIGYSDSAKDAGVMAASWAQYQAQDALIKTCEKAGIELTLFHGRGGSIGRGGAPAHAALLSQPPGSLKGGLRVTEQGEMIRFKYGLPEITVSSLSLYTGAILEANLLPPPEPKESWRRIMDELSVISCDVYRGYVRENKDFVPYFRSATPEQELGKLPLGSRPAKRRPTGGVESLRAIPWIFAWTQNRLMLPAWLGAGTALQKVVEDGKQSELEAMCRDWPFFSTRLGMLEMVFAKADLWLAEYYDQRLVDKALWPLGKELRNLQEEDIKVVLAIANDSHLMADLPWIAESIQLRNIYTDPLNVLQAELLHRSRQAEKEGQEPDPRVEQALMVTIAGIAAGMRNTG

SEQ ID NO: 9 - protein, 575 aa, Pichia kudriavzevii
MTDKISLGTYLFEKLKEAGSYSIFGVPGDFNLALLDHVKEVEGIRWVGNANELNAGYEADGYARINGFASLITTFGVGELSAVNAIAGSYAEHVPLIHIVGMPSLSAMKNNLLLHHTLGDTRFDNFTEMSKKISAKVEIVYDLESAPKLINNLIETAYHTKRPVYLGLPSNFADELVPAALVKENKLHLEEPLNNPVAEEEFIHNVVEMVKKAEKPIILVDACAARHNISKEVRELAKLTKFPVFTTPMGKSTVDEDDEEFFGLYLGSLSAPDVKDIVGPTDCILSLGGLPSDFNTGSFSYGYTTKNVVEFHSNYCKFKSATYENLMMKGAVQRLISELKNIKYSNVSTLSPPKSKFAYESAKVAPEGIITQDYLWKRLSYFLKPRDIIVTETGTSSFGVLATHLPRDSKSISQVLWGSIGFSLPAAVGAAFAAEDAHKQTGEQERRTVLFIGDGSLQLTVQSISDAARWNIKPYIFILNNRGYTIEKLIHGRHEDYNQIQPWDHQLLLKLFADKTQYENHVVKSAKDLDALMKDEAFNKEDKIRVIELFLDEFDAPEILVAQAKLSDEINSKAA

SEQ ID NO: 10 - protein, 563 aa, Saccharomyces cerevisiae
MSEITLGKYLFERLKQVNVNTVFGLPGDFNLSLLDKIYEVEGMRWAGNANELNAAYAADGYARIKGMSCIITTFGVGELSALNGIAGSYAEHVGVLHVVGVPSISAQAKQLLLHHTLGNGDFTVFHRMSANISETTAMITDIATAPAEIDRCIRTTYVTQRPVYLGLPANLVDLNVPAKLLQTPIDMSLKPNDAESEKEVIDTILALVKDAKNPVILADACCSRHDVKAETKKLIDLTQFPAFVTPMGKGSIDEQHPRYGGVYVGTLSKPEVKEAVESADLILSVGALLSDFNTGSFSYSYKTKNIVEFHSDHMKIRNATFPGVQMKFVLQKLLTTIADAAKGYKPVAVPARTPANAAVPASTPLKQEWMWNQLGNFLQEGDVVIAETGTSAFGINQTTFPNNTYGISQVLWGSIGFTTGATLGAAFAAEEIDPKKRVILFIGDGSLQLTVQEISTMIRWGLKPYLFVLNNDGYTIEKLIHGPKAQYNEIQGWDHLSLLPTFGAKDYETHRVATTGEWDKLTQDKSFNDNSKIRMIEIMLPVFDAPQNLVEQAKLTAATNAKQ

SEQ ID NO: 11 - protein, 376 aa, Pichia kudriavzevii
MFASTFRSQAVRAARFTRFQSTFAIPEKQMGVIFETHGGPLQYKEIPVPKPKPTEILINVKYSGVCHTDLHAWKGDWPLPAKLPLVGGHEGAGIVVAKGSAVTNFEIGDYAGIKWLNGSCMSCEFCEQGDESNCEHADLSGYTHDGSFQQYATADAIQAAKIPKGTDLSEVAPILCAGVTVYKALKTADLRAGQWVAISGAAGGLGSLAVQYAKAMGLRVLGIDGGEGKKELFEQCGGDVFIDFTRYPRDAPEKMVADIKAATNGLGPHGVINVSVSPAAISQSCDYVRATGKVVLVGMPSGAVCKSDVFTHVVKSLQIKGSYVGNRADTREALEFFNEGKVRSPIKVVPLSTLPEIYELMEQGKILGRYVVDTSK

SEQ ID NO: 12 - protein, 388 aa, Pichia kudriavzevii
MVSPAERLSTIASTIKPNRKDSTSLQPEDYPEHPFKVTVVGSGNWGCTIAKVIAENTVERPRQFQRDVNMWVYEELIEGEKLTEIINTKHENVKYLPGIKLPVNVVAVPDIVEACAGSDLIVFNIPHQFLPRILSQLKGKVNPKARAISCLKGLDVNPNGCKLLSTVITEELGIYCGALSGANLAPEVAQCKWSETTVAYTIPDDFRGKGKDIDHQILKSLFHRPYFHVRVISDVAGISIAGALKNVVAMAAGFVEGLGWGDNAKAAVMRIGLVETIQFAKTFFDGCHAATFTHESAGVADLITTCAGGRNVRVGRYMAQHSVSATEAEEKLLNGQSCQGIHTTREVYEFLSNMGRTDEFPLFTTTYRIIYENFPIEKLPECLEPVED

SEQ ID NO: 13 - protein, 342 aa, Pichia kudriavzevii
MSNVKVALLGAAGGIGQPLALLLKLNPNITHLALYDVVHVPGVAADLHHIDTDVVITHHLKDEDGTALANALKDATFVIVPAGVPRKPGMTRGDLFTINAGICAELANAISLNAPNAFTLVITNPVNSTVPIFKEIFAKNEAFNPRRLFGVTALDHVRSNTFLSELIDGKNPQHFDVTVVGGHSGNSIVPLFSLVKAAENLDDEIIDALIHRVQYGGDEVVEAKSGAGSATLSMAYAANKFFNILLNGYLGLKKTMISSYVFLDDSINGVPQLKENLSKLLKGSEVELPTYLAVPMTYGKEGIEQVFYDWVFEMSPKEKENFITAIEYIDQNIEKGLNFMVR

SEQ ID NO: 14 - protein, 267 aa, Artificial Sequence
  Bacterial consensus sequence
MLHIAMIGCGAIGAGVLELLKSDPDLRVDAVIVPEESMDAVREAVAALAPVARVLTALPADARPDLLVECAGHRAIEEHVVPALERGIPCAVASVGALSEPGLAERLEAAARRGGTQVQLLSGAIGAIDALAAARVGGLDSVVYTGRKPPLAWKGTPAEQVCDLDALTEATVIFEGSAREAARLYPKNANVAATLSLAGLGLDRTQVRLIADPAVTENVHHVEARGAFGGFELTMRGKPLAANPKTSALTVYSVVRALGNRAHALSI

SEQ ID NO: 15 - protein, 128 aa, Artificial Sequence
  Bacterial consensus sequence
MLRTMLKSKIHRATVTQADLHYVGSVTIDADLLDAADILEGEKVAIVDITNGARLETYVIAGERGSGVIGINGAAAHLVHPGDLVIIIAYAQMSDAEARAYEPRVVFVDADNRIVELGNDPAEALPGG

SEQ ID NO: 16 - protein, 585 aa, Artificial Sequence
  Eukaryotic consensus sequence
MPANGNFPVALEVISIFKPYNSAVEDLASMAKTDTSASSSGSDSAGSSEDEDVQLFASKGNLLNSKLLKKSNNNNKNNNINENNNKNAAAGLKRFASLPNRAEHEEFLRDCVDEILKLAVFEGTNRSSKVVEWHDPEELKKLFDFELRAEPDSHEKLLELLRATIRYSVKTGHPYFVNQLFSSVDPYGLVGQWLTDALNPSVYTYEVAPVFTLMEEVVLREMRRIVGFPNDGEGDGIFCPGGSIANGYAISCARYKYAPEVKKKGLHSLPRLVIFTSEDAHYSVKKLASFMGIGSDNVYKIATDEVGKMRVSDLEQEILRALDEGAQPFMVSATAGTTVIGAFDPLEGIADLCKKYNLWMHVDAAWGGGALMSKKYRHLLKGIERADSVTWNPHKLLAAPQQCSTFLTRHEGILSECHSTNATYLFQKDKFYDTSYDTGDKHIQCGRRADVLKFWFMWKAKGTSGFEAHVDKVFENAEYFTDSIKARPGFELVIEEPECTNICFWYVPPSLRGMERDNAEFYEKLHKVAPKIKERMIKEGSMMITYQPLRDLPNFFRLVLQNSGLDKSDMLYFINEIERLGSDLV

SEQ ID NO: 17 - protein, 268 aa, Ralstonia solanacearum
MLHVSMVGCGAIGQGVLELLKSDPDLCFDTVIVPEHGMDRARAAIAPFAPRTRVMTRLPAQADRPDLLVECAGHDALREHVVPALEQGIDCLVVSVGALSEPGLAERLEAAARRGHAQMQLLSGAIGAIDALAAARVGGLDAVVYTGRKPPRAWKGTPAERQFDLDALDRTTVIFEGKASDAALLFPKNANVAATLALAGLGMERTHVRLLADPTIDENIHHVEARGAFGGFELIMRGKPLAANPKTSALTVFSVVRALGNRAHAVSI

SEQ ID NO: 18 - protein, 267 aa, Polaromonas species
MLKIAMIGCGAIGASVLELLHGDSDVVVDRVITVPEARDRTEIAVARWAPRARVLEVLAADDAPDLVVECAGHGAIAAHVVPALERGIPCVVTSVGALSAPGMAQLLEQAARRGKTQVQLLSGAIGGIDALAAARVGGLDSVVYTGRKPPMAWKGTPAEAVCDLDSLTVAHCIFDGSAEQAAQLYPKNANVAATLSLAGLGLKRTQVQLFADPGVSENVHHVAAHGAFGSFELTMRGRPLAANPKTSALTVYSVVRALLNRGRALVI

SEQ ID NO: 19 - protein, 271 aa, Burkholder thailandensis
MRNAHAPVDVAMIGFGAIGAAVYRAVEHDAALRVAHVIVPEHQCDAVRGALGERVDVVSSVDALAYRPQFALECAGHGALVDHVVPLLRAGTDCAVASIGALSDLALLDALSEAADEGGATLTLLSGAIGGVDALAAAKQGGLDEVQYIGRKPPLGWLGTPAEALCDLRAMTAEQTIFEGSARDAARLYPKNANVAATVALAGVGLDATKVRLIADPAVTRNVHRVVARGAFGEMSIEMSGKPLPDNPKTSALTAFSAIRALRNRASHCVI

SEQ ID NO: 20 - protein, 271 aa, Burkholderia pseudomallei
MRNAHAPVDVAMIGFGAIGAAVYRAVEHDAALRVAHVIVPEHQCDAVRGALGERVDVVSSVDALACRPQFALECAGHGALVDHVVPLLKAGTDCAVASIGALSDLALLDALSNAADAGGATLTLLSGAIGGIDALAAARQGGLDEVRYIGRKPPLGWLGTPAEAICDLRAMAAEQTIFEGSARDAAQLYPRNANVAATIALAGVGLDATRVCLIADPAVTRNVHRIVARGAFGEMSIEMSGKPLPDNPKTSALTAFSAIRALRNRASHCVI

SEQ ID NO: 21 - protein, 268 aa, Ochrobactrum anthropi
MSVSETIVLVGWGAIGKRVADLLAERKSSVRIGAVAVRDRSASRDRLPAGAVLIENPAELAASGASLVVEAAGRPSVLPWGEAALSTGMDFAVSSTSAFVDDALFQRLKDAAAASGAKLIIPPGALGGIDALSAASRLSIESVEHRIIKPAKAWAGTQAAQLVPLDEISEATVFFTDTARKAADAFPQNANVAVITSLAGIGLDRTRVTLVADPAARLNTHEIIAEGDFGRMHLRFENGPLATNPKSSEMTALNLVRAIENRVATTVI

SEQ ID NO: 22 - protein, 263 aa, Acinetobacter species
MKKLMMIGFGAMAAEVYAHLPQDLQLKWIVVPSRSIEKVQSQVSSEIQVISDIEQCDGTPDYVIEVAGQAAVKEHAQKVLAKGWTIGLISVGTLADSEFLIQLKQTAEKNDAHLHLLAGAIAGIDGISAAKEGGLQKVTYKGCKSPKSWKGSYAEQLVDLDHVVEATVFFTGTAREAATKFPANANVAATIALAGLGMDETMVELTVDPTINKNKHTIVAEGGFGQMTIELVGVPLPSNPKTSTLAALSVIRACRNSVEAIQI

SEQ ID NO: 23 - protein, 255 aa, Klebsiella pneumoniae
MMKKVMLIGYGAMAQAVIERLPPQVRVEWIVARESHHAAICLQFGQAVTPLTDPLQCGGTPDLVLECASQQAVAQYGEAVLARGWHLAVISTGALADSELEQRLRQAGGKLTLLAGAVAGIDGLAAAKEGGLERVTYQSRKSPASWRGSYAEQLIDLSAVNEAQIFFEGSAREAARLFPANANVAATIALGGIGLDATRVQLMVDPATQRNTHTLHAEGLFGEFHLELSGLPLASNPKTSTLAALSAVRACRELA

SEQ ID NO: 24 - protein, 253 aa, Dinoroseobacter shibae
MRLALIGLGAINRAVAAGMAGQAEMVALTRSGAEAPGVMAVSDLSALRVFAPDLVVEAAGHGAARAYLPGLLAAGIDVLMASVGVLADPETEAAFRAAPAHGAQLTIPAGAIGGLDLLAALPKDSLRAVRYTGVKPPAAWAGSPAADGRDLSALDGPVTLFEGTARQAALRFPNNANVAATLALAGAGFDRTEARLVADPDAAGNGHAYDVISDTAEMTFSVRARPSDTPGTSATTAMSLLRAIRNRDAAWVV

SEQ ID NO: 25 - protein, 275 aa, Ruegeria pomeroyi
MWKLWGSWPEGDRVRIALIGHGPIAAHVAAHLPVGVQLTGALCRPGRDDAARAALGVSVAQALEGLPQRPDLLVDCAGHSGLRAHGLTALGAGVEVLTVSVGALADAVFCAELEDAARAGGTRLCLASGAIGALDALAAAAMGTGLQVTYTGRKPPQGWRGSRAEKVLDLKALTGPVTHFTGTARAAAQAYPKNANVAAAVALAGAGLDATRAELIADPGAAANIHEIAAEGAFGRFRFQIEGLPLPGNPRSSALTALSLLAALRQRGAAIRPSF

SEQ ID NO: 26 - protein, 266 aa, Comamonas testosteroni
MKNIALIGCGAIGSSVLELLSGDTQLQVGWVLVPEITPAVRETAARLAPQAQLLQALPGDAVPDLLVECAGHAAIEEHVLPALARGIPAVIASIGALSAPGMAERVQAAAETGKTQAQLLSGAIGGIDALAAARVGGLETVLYTGRKPPKAWSGTPAEQVCDLDGLTEAFCIFEGSAREAAQLYPKNANVAATLSLAGLGLDKTMVRLFADPGVQENVHQVEARGAFGAMELTMRGKPLAANPKTSALTVYSVVRAVLNNVAPLAI

SEQ ID NO: 27 - protein, 268 aa, Cupriavidius pinatubonensis
MSMLHVSMVGCGAIGRGVLELLKADPDVAFDVVIVPEGQMDEARSALSALAPNVRVATGLDGQRPDLLVECAGHQALEEHIVPALERGIPCMVVSVGALSEPGLVERLEAAARRGNTQVQLLSGAIGAIDALAAARVGGLDEVIYTGRKPARAWTGTPAAELFDLEALTEPTVIFEGTARDAARLYPKNANVAATVSLAGLGLDRTSVRLLADPNAVENVHHIEARGAFGGFELTMRGKPLAANPKTSALTVFSVVRALGNRAHAVSI